(19) United States
(12) Patent Application Publication
(10) Pub. No.: US 2026/0253707 A1
(43) Pub. Date: Aug. 27, 2026

(54) SYSTEM

(71) Applicant: SoftBank Group Corp., Tokyo (JP)

(72) Inventor: Shinnosuke MORITA, Tokyo (JP)

(73) Assignee: SoftBank Group Corp., Tokyo (JP)

(21) Appl. No.: 19/534,783

(22) Filed: Feb. 10, 2026

(30) Foreign Application Priority Data
Feb. 21, 2025 (JP) 2025-027029

(51) Int. Cl. G16H 20/60; G16H 50/20; G16H 50/30; G16H 50/70

(52) U.S. Cl. CPC G16H 20/60; G16H 50/20; G16H 50/30; G16H 50/70

(57) ABSTRACT

The system according to the embodiment comprises a receiving unit, an analysis unit, a determination unit, and a proposal unit. The receiving unit uploads genetic test results. The analysis unit analyzes the genetic test results uploaded by the receiving unit and questionnaire information. The determination unit determines disease risk based on information analyzed by the analysis unit. The proposal unit proposes a meal kit for reducing the disease risk determined by the determination unit.

### CROSS-REFERENCE TO RELATED APPLICATION

The present application claims priority to and incorporates by reference the entire contents of Japanese Patent Application No. 2025-027029 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, there has been a problem that determination of disease risk based on genetic test results and proposals for specific countermeasures based on such determination have not been sufficiently performed.

### SUMMARY OF THE INVENTION

The system according to the embodiment comprises a receiving unit, an analysis unit, a determination unit, and a proposal unit. The receiving unit uploads genetic test results. The analysis unit analyzes the genetic test results uploaded by the receiving unit and questionnaire information. The determination unit determines disease risk based on information analyzed by the analysis unit. The proposal unit proposes a meal kit for reducing the disease risk determined by the determination unit.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

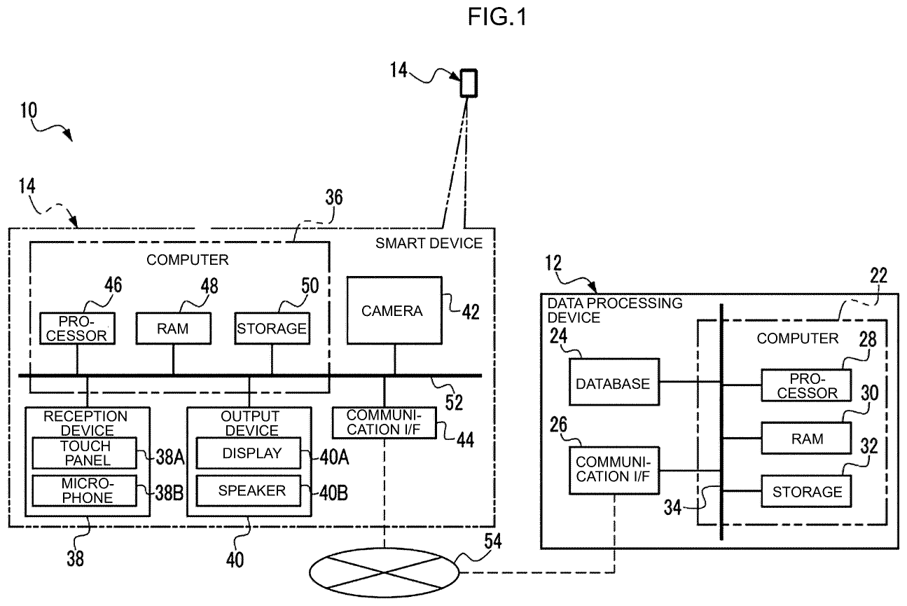

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

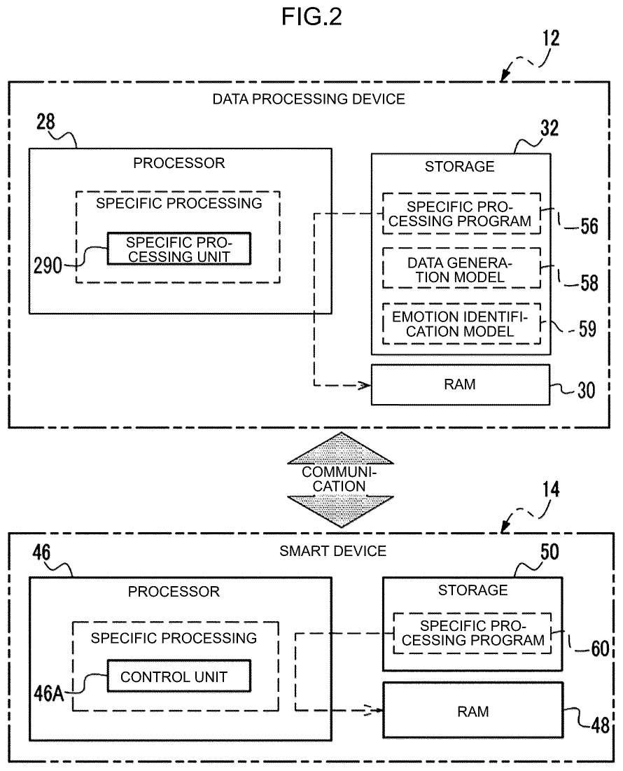

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example of the Embodiment

The system according to the embodiment of the present invention is a system in which a user undergoes a human genetic test and uploads the results to a generative AI, thereby periodically delivering a meal kit to reduce disease risk. In this system, the user undergoes a human genetic test, uploads the results to a generative AI, and enters a simple questionnaire regarding the user's current health status and lifestyle habits, so that a meal kit to reduce disease risk is proposed and delivered regularly. For example, the user collects a sample such as saliva or blood using a genetic test kit and sends it to a testing institution. The testing institution analyzes the status of approximately 21,000 genetic loci of the user's genetic set and provides the results. Next, the user uploads the genetic test results to the generative AI. The generative AI analyzes the uploaded genetic test results and grasps the user's genetic information. Furthermore, the user enters a simple questionnaire regarding current health status and lifestyle habits. The generative AI analyzes the questionnaire information and grasps the user's lifestyle habits. The generative AI analyzes the uploaded genetic test results and questionnaire information to determine the user's disease risk. For example, if a specific gene increases the risk of disease, the system can propose improvements in diet or lifestyle habits to reduce that risk. Finally, the generative AI proposes a meal kit to reduce the determined disease risk and delivers it regularly. The meal kit includes nutritionally balanced meals based on the user's genetic information and lifestyle habits. This enables the user to manage their health individually based on their genetic information and reduce disease risk. Thus, the system provides individualized health management based on the user's genetic information and can reduce disease risk. Specifically, the system accepts as input data the human genetic test result data obtained by the user (for example, CSV files or JSON format data including SNP sequence information and allele type information for 22,000 genetic loci). The user enters health status and lifestyle habits (e.g., daily steps, sleep duration, smoking/drinking habits, meal frequency, stress level, etc.) in a questionnaire format, which are preprocessed as numerical vectors or categorical data. As the generative AI, a Transformer-based large language model, a multilayer perceptron specialized for genetic data analysis, or a multimodal neural network can be used. The AI model integrates genetic sequence information (e.g., allele type vectors for each rsID, numerical data such as 0/1/2), lifestyle habit vectors (e.g., sleep duration=7.5, steps=8000, drinking=1, etc.), and past disease history or family history as input tensors. From these inputs, the AI model outputs disease risk scores for each disease (e.g., type 2 diabetes, dyslipidemia, hypertension, etc. ; probability values such as 0.85, 0.32, etc. between 0 and 1) and contribution rates for each risk factor (e.g., gene A=0.12, lifestyle habit B=0.08, etc.). Example outputs include “Type 2 diabetes risk 0.78, main factors: rs1234567 (T/T), high-fat diet, lack of sleep” or “Hypertension risk 0.45, main factors: rs9876543 (C/G), lack of exercise.” Based on these risk scores, the AI model automatically generates the contents of the meal kit (e.g., low-carb menu, omega-3 fatty acid enhancement, low-salt diet, etc.) and optimizes them considering the user's preferences and allergy information. In subsequent processing, the AI output is used for threshold determination (e.g., intensive intervention for risk above 0.7), data linkage to the proposal unit, and instructions to the delivery scheduler. As a technical effect, this system, unlike conventional uniform health guidance or manual meal proposals, can integrate and analyze vast genetic and lifestyle data in high-dimensional space and automatically generate individualized intervention measures in real time, thereby dramatically improving the accuracy and efficiency of health management. Furthermore, for AI model training, loss functions such as cross-entropy or MSE are used, and weight optimization is performed by gradient descent, enabling continuous improvement of determination accuracy. Application fields include personal health management services, risk assessment for insurance companies, employee health support for companies, and public health measures for local governments.

The system according to the embodiment comprises a receiving unit, an analysis unit, a determination unit, and a proposal unit. The receiving unit uploads genetic test results obtained by the user. For example, the user collects a sample such as saliva or blood using a genetic test kit and sends it to a testing institution. The testing institution analyzes the status of approximately 21,000 genetic loci of the user's genetic set and provides the results. The receiving unit uploads the genetic test results received by the user to the generative AI. The analysis unit analyzes the uploaded genetic test results and questionnaire information entered by the user. For example, the analysis unit analyzes whether specific genes increase the risk of disease. The analysis unit analyzes questionnaire information regarding the user's current health status and lifestyle habits and grasps the user's lifestyle habits. The determination unit determines disease risk based on information analyzed by the analysis unit. For example, if a specific gene increases the risk of disease, the determination unit determines improvements in diet or lifestyle habits to reduce that risk. The proposal unit proposes a meal kit to reduce the disease risk determined by the determination unit. For example, the proposal unit proposes nutritionally balanced meals based on the user's genetic information and lifestyle habits. The proposal unit can also propose a meal kit customizable according to the user's preferences. Thus, the system according to the embodiment provides individualized health management based on the user's genetic information and can reduce disease risk. Specifically, the system receives genetic test data in CSV or JSON format (e.g., SNP sequences and allele type information for 22,000 genetic loci) from the user terminal via the receiving unit and stores it in a database. The receiving unit performs data integrity verification (e.g., duplicate check of rsID, missing value completion, anomaly detection) and secure data transfer via encrypted communication. The analysis unit vectorizes the received data, generating allele type vectors for each rsID in 0/1/2 representation and numerical vectors for lifestyle questionnaire data (e.g., sleep duration=7.0, steps=9000, drinking=0, etc.). The analysis unit inputs these vectors into a Transformer-based large language model, multilayer perceptron, or multimodal neural network. The AI model integrates genetic information and lifestyle information in high-dimensional space and outputs disease risk scores for each disease (e.g., type 2 diabetes risk 0.82, dyslipidemia risk 0.41, etc.) and contribution rates for each risk factor (e.g., rs1234567=0.13, lack of sleep=0.09, etc.). The determination unit performs threshold determination (e.g., high risk for scores above 0.7) and rule-based determination based on combinations of risk factors (e.g., specific genotype plus high-fat diet increases risk) on the AI model's output scores. The determination unit can also apply multivariate logistic regression or decision tree algorithms considering user attributes (age, gender, family history, etc.). The proposal unit automatically generates the contents of the meal kit (e.g., low-carb menu, omega-3 fatty acid enhancement, low-salt diet, etc.) based on the determination results and customizes them reflecting the user's preferences and allergy information. The proposal unit can also implement ingredient combination optimization algorithms (e.g., linear programming, genetic algorithms) and recommendation systems utilizing past user satisfaction data. As a technical effect, this system, unlike conventional manual and uniform health guidance or meal proposals, can integrate and analyze vast genetic and lifestyle data in high-dimensional space and automatically generate individualized intervention measures in real time, thereby dramatically improving the accuracy and efficiency of health management. For AI model training, cross-entropy loss or MSE is used, and weight optimization is performed by gradient descent, enabling continuous improvement of determination accuracy. Application fields include personal health management services, risk assessment for insurance companies, employee health support for companies, and public health measures for local governments.

The system comprises a voice input unit that enables questionnaire input by voice. The voice input unit allows the user to enter questionnaire responses by voice. For example, the voice input unit supports the user in entering questionnaire answers by voice using a microphone. The voice input unit uses voice recognition technology to convert the user's voice into text data. For example, the voice input unit enables the user to answer questions such as “Please tell me about today's meals” by voice. The voice input unit analyzes the user's voice in real time and automatically enters questionnaire responses. Thus, the voice input unit enables the user to easily enter questionnaire responses. Specifically, the system accepts audio waveform data (e.g., 16 kHz, 16 bit PCM format, single-channel time series) obtained from the microphone of the user terminal as input data for the voice input unit. The voice input unit first performs preprocessing such as noise reduction and volume normalization, and then uses a voice recognition engine (e.g., Transformer-based end-to-end speech recognition model or CTC-based deep neural network) to convert the audio waveform into a token sequence or string data. Example inputs include natural language speech such as “I ate fish yesterday” or “I walked 8,000 steps in a day.” The speech recognition model extracts acoustic features (e.g., Mel spectrogram, MFCC, etc.), learns time series features in the encoder layer, and outputs text sequences in the decoder layer. The output data is structured as text answers for each questionnaire item (e.g., “meal=fish,” “steps=8000,” etc.). After speech recognition, a natural language processing module extracts intent and entities (e.g., ingredient names, quantities, frequencies, etc.) and converts them into numerical vectors or categorical data. Subsequently, these data are linked to the analysis unit and integrated with genetic information and lifestyle information to be incorporated into the input tensor of the health risk determination AI model. As a technical effect, this system, compared to conventional manual or form input, enables fast and intuitive data acquisition by voice, greatly improving usability, and the introduction of speech recognition AI allows flexible handling of diverse speech patterns and dialects, dramatically improving input accuracy and convenience. Furthermore, by accumulating voice input history, applying custom acoustic models tailored to each user's speech tendencies, and implementing automatic correction algorithms for misrecognition, continuous improvement of recognition accuracy is possible. Application fields include health management applications, medical interview support, senior healthcare services, and integration with wearable devices.

The system comprises a visualization unit that visualizes analysis results. The visualization unit visually displays analysis results. For example, the visualization unit can display analysis results as graphs or charts. The visualization unit displays analysis results with color coding to make it easier for the user to understand the results. For example, the visualization unit displays genes with high disease risk in red and genes with low risk in green. The visualization unit can also display analysis results interactively. For example, the visualization unit allows the user to click on graphs or charts to display detailed information. Thus, the visualization unit enables the user to intuitively understand analysis results. Specifically, the system accepts health risk scores and gene contribution data output from the analysis unit or determination unit (e.g., risk probability values for each disease, contribution vectors for each genetic locus, weights of lifestyle factors, etc.) as input data for the visualization unit. The visualization unit converts these numerical data into various visualization formats such as bar graphs, pie charts, heat maps, and radar charts using graphic libraries such as D3.js or WebGL. Example inputs include structured data such as “Type 2 diabetes risk=0.78, main factors: rs1234567 (T/T), high-fat diet” or “Hypertension risk=0.45, main factors: rs9876543 (C/G), lack of exercise.” The visualization unit automatically adjusts hue and brightness according to risk values, highlights threshold-exceeding areas, and provides interaction functions such as pop-up display of detailed explanations or historical trend graphs for relevant genes or lifestyle factors when the user clicks or taps on graph elements. Furthermore, the visualization unit optimizes the layout responsively according to the user's device (smartphone, tablet, PC, etc.) and screen size, and can implement accessibility features such as color vision diversity and voice readout. Subsequently, the output of the visualization unit is linked to feedback modules for improving user understanding and promoting behavioral change, or to report generation functions for medical professionals. As a technical effect, this system, compared to conventional text-based presentation of analysis results, enables intuitive and interactive visualization of complex high-dimensional data, greatly improving user understanding and acceptance, and increasing the rate of health behavior implementation. In addition, automatic layout optimization by AI and personalized display based on user operation history enable continuous UX improvement and enhancement of analysis accuracy. Application fields include personal health management dashboards, patient explanation support in medical institutions, risk assessment reports for insurance companies, and public health monitoring for local governments.

The system comprises an improvement unit that improves proposed content based on user feedback. The improvement unit collects user feedback and improves the proposed content. For example, the improvement unit analyzes feedback provided by the user and identifies points for improvement in the proposed content. The improvement unit automatically updates the proposed content based on user feedback. For example, if the user provides feedback such as “I want more variety in the meal kit,” the improvement unit updates the proposed content to propose a more varied meal kit. The improvement unit accumulates user feedback and utilizes it to improve the proposed content. Thus, the improvement unit can provide proposed content tailored to user needs. Specifically, the system accepts feedback data collected from users (e.g., free-text comments, five-point ratings, multiple-choice questionnaires, meal kit satisfaction scores, etc.) as input data for the improvement unit. The improvement unit uses a natural language processing AI (e.g., Transformer-based large language model) to extract key phrases such as requests, complaints, and points for improvement, as well as emotion scores, from free-text feedback and structure the data. Example inputs include text such as “I want more vegetables,” “I would be happy with more Japanese-style menus,” or numerical data such as “satisfaction=3,” “variation=2.” The improvement unit classifies these data by clustering or topic modeling and identifies frequent requests or complaints. Furthermore, by using AI-based recommendation algorithms (e.g., collaborative filtering, content-based recommendation), the improvement unit matches user attributes and past feedback history and automatically adjusts the meal kit generation parameters of the proposal unit (e.g., ingredient variation, cooking method, nutritional balance, etc.). Subsequently, the output of the improvement unit is linked to the proposal unit and reflected in the contents of the meal kit and proposal text for subsequent deliveries. As a technical effect, this system, unlike conventional one-way proposals or manual feedback aggregation, enables continuous realization of personalized proposals that promptly respond to individual user needs and changes in preferences through automatic AI analysis and real-time reflection, thereby greatly improving user satisfaction and continued usage rate. In addition, accumulation of feedback data increases the training data for the AI model, enabling continuous improvement of proposal accuracy. Application fields include health food subscription services, personalized meal systems, corporate welfare support, and health promotion measures for local governments.

The receiving unit can automatically upload genetic test results. The receiving unit is equipped with a function to automatically upload genetic test results to eliminate the need for the user to manually upload them. For example, the receiving unit automatically uploads genetic test results to the generative AI when the results are saved on the user's device. The receiving unit receives a notification when the user receives genetic test results and automatically starts uploading. Thus, the receiving unit eliminates the need for the user to manually upload genetic test results and enables smooth progress of the analysis process. Specifically, the system monitors the file system or cloud storage of the user terminal as the receiving unit and detects events such as new saving or updating of genetic test result files (e.g., CSV, JSON, VCF files containing SNP sequence data for 22,000 genetic loci). The receiving unit automatically verifies the file contents (e.g., file format integrity, duplicate/missing check of rsID, virus scan, etc.) and securely uploads them to the server-side AI analysis platform using encrypted communication (e.g., TLS/SSL). Example inputs include files such as “genome_result_20240601.csv” or “user123_snp.json.” After upload completion, the receiving unit returns a notification to the user terminal and automates instructions for resending in case of errors or progress updates. Subsequently, the uploaded data is linked to the analysis unit and integrated with lifestyle questionnaire and voice input data to be incorporated into the input tensor of the AI model. As a technical effect, this system eliminates the risks of data loss or delay due to manual upload or user operation errors, automates the entire process from data acquisition to analysis, greatly reducing user burden and improving real-time performance and data quality. In addition, accumulation of upload history and error occurrence logs contributes to improved system reliability and rapid identification of causes in case of failure. Application fields include personal genetic testing services, automatic data linkage for medical institutions, automated risk assessment for insurance companies, and health data collection platforms for local governments.

The analysis unit can analyze genetic information and questionnaire information to grasp the user's lifestyle habits. By analyzing genetic information and questionnaire information, the analysis unit can grasp the user's lifestyle habits in detail. For example, the analysis unit analyzes the user's dietary patterns, exercise habits, and sleep patterns. The analysis unit determines, based on the user's genetic information, whether specific lifestyle habits increase the risk of disease. For example, the analysis unit analyzes whether the user's consumption of a high-fat diet is associated with specific genetic mutations. Thus, the analysis unit can grasp the user's lifestyle habits in detail and propose appropriate health management. Specifically, the system accepts genetic test data (e.g., SNP sequence vectors for 22,000 genetic loci, 0/1/2 representation for each rsID) and questionnaire information (e.g., numerical vectors or categorical data for meal frequency, exercise amount, sleep duration, drinking/smoking habits, etc.) as input data for the analysis unit. The analysis unit preprocesses these data, performs missing value completion and normalization, and inputs them into a multimodal neural network (e.g., integrated model of MLP for genetic information and Transformer for lifestyle habits). The AI model integrates genetic information and lifestyle information in high-dimensional space and estimates the contribution of each lifestyle factor to disease risk and the interaction effects between specific genotypes and lifestyle habits (e.g., rs1234567 is T/T type and high-fat diet increases risk). Example inputs include “rs1234567=2, rs9876543=1, diet=high-fat, steps=8000, sleep=6.5 h.” The AI model outputs risk scores for each disease (e.g., type 2 diabetes risk=0.81, dyslipidemia risk=0.39, etc.) and contribution vectors for each risk factor (e.g., high-fat diet=0.12, rs1234567=0.09, etc.). Subsequently, these outputs are linked to the determination unit and proposal unit and used as supporting data for individualized health management proposals. As a technical effect, this system, unlike conventional simple questionnaire aggregation or manual lifestyle evaluation, can integrate and analyze vast genetic and diverse lifestyle data with AI to automatically generate optimized risk assessments and intervention proposals for each user in real time, thereby dramatically improving the accuracy and efficiency of health management. Furthermore, for AI model training, cross-entropy loss and gradient descent are used, enabling continuous improvement of determination accuracy. Application fields include personal health management services, risk assessment for insurance companies, employee health support for companies, and public health measures for local governments.

The proposal unit can propose a meal kit customizable according to the user's preferences. The proposal unit is equipped with a function to customize meal kits according to the user's preferences. For example, if the user prefers specific ingredients, the proposal unit proposes a meal kit containing those ingredients. If the user has allergies to specific ingredients, the proposal unit proposes a meal kit excluding those ingredients. The proposal unit considers the user's preferences and allergy information to propose the optimal meal kit. Thus, the proposal unit can improve user satisfaction. Specifically, the system accepts user preference data (e.g., list of favorite ingredients, list of disliked ingredients, allergy information, past meal kit evaluation scores, etc.) as input data for the proposal unit. The proposal unit inputs this information into a meal kit generation algorithm (e.g., nutritional balance optimization by linear programming, ingredient combination search by genetic algorithm, collaborative filtering recommendation model, etc.) and automatically generates optimized meal kit contents for each user. Example inputs include “favorite ingredients=chicken, tomato,” “allergy=wheat,” “past evaluation=Japanese food 4 points, Western food 2 points,” etc. The proposal unit links with genetic information and lifestyle data, prioritizes ingredients and cooking methods that contribute to disease risk reduction, and strictly reflects preference and allergy constraints. Example outputs include “low-carb Japanese-style menu with chicken and tomato as main ingredients” or “gluten-free meal kit without wheat.” Subsequently, the proposed content is linked to the delivery scheduler and user notification module and used for regular delivery and proposal text generation. As a technical effect, this system, unlike conventional uniform meal proposals or manual customization, can automatically generate individualized proposals in real time that simultaneously satisfy user preferences, constraints, and health risks by introducing AI-based multivariate optimization and recommendation algorithms, thereby greatly improving user satisfaction and continued usage rate. Furthermore, by accumulating preference data and evaluation history and utilizing them as training data for the AI model, continuous improvement of proposal accuracy is possible. Application fields include personalized health food services, allergy-friendly meal systems, corporate welfare support, and health promotion measures for local governments.

The receiving unit can estimate the user's emotions and adjust the timing of uploading genetic test results based on the estimated emotions. The receiving unit estimates the user's emotions and adjusts the timing of uploading genetic test results based on the estimated emotions. For example, if the user is feeling stressed, the receiving unit prompts uploading during a relaxing time. If the user is busy, the receiving unit adjusts so that uploading can be completed quickly. If the user is relaxed, the receiving unit provides detailed explanations during uploading. Emotion estimation is realized by using an emotion estimation function with, for example, an emotion engine or generative AI. The generative AI may be a text generative AI (e.g., LLM) or a multimodal generative AI, but is not limited to these examples. Thus, the receiving unit can upload genetic test results at the optimal timing according to the user's emotions. Specifically, the receiving unit accepts as input data voice data obtained from the user terminal (e.g., 16 kHz, 16 bit PCM format audio waveform), text input (e.g., natural language sentences such as “I'm busy today,” “I'm calm now”), or facial images (e.g., facial expression images taken by camera, 224×224 pixel RGB images) for emotion estimation AI. As the emotion estimation AI, a multimodal neural network combining a Transformer-based model for voice recognition, CNN for facial expression recognition, and a large language model for text emotion classification can be used. Example inputs include “voice: speech with sighs,” “text: very busy today,” “image: smiling face.” The AI model extracts acoustic features (e.g., MFCC, pitch, energy), text embedding vectors (e.g., 768-dimensional BERT-based vectors), and image feature maps (e.g., 512-dimensional ResNet output vectors), and outputs emotion categories (e.g., stress, relaxation, excitement, fatigue, etc.) and emotion scores (e.g., stress level 0.82, relaxation level 0.15, etc.) in the integration layer. Example outputs include “stress=0.75, relaxation=0.10,” “emotion label=stress,” “emotion label=relaxation.” Based on these emotion estimation results, the receiving unit executes an upload timing control algorithm (e.g., notification at night or on holidays when stress level is high, immediate upload promotion when relaxation level is high, one-tap upload UI when busyness is high). Subsequently, the determination of upload timing is linked to the notification scheduler for the user terminal, automatic switching of upload UI, and instructions to the explanation text generation module. As a technical effect, this receiving unit, unlike conventional uniform upload timing presentation or user-dependent operation, can reduce the user's psychological burden and greatly improve upload completion rate and data quality by multimodal emotion estimation and dynamic timing control by AI. Furthermore, for emotion estimation AI training, cross-entropy loss and data augmentation (e.g., adding noise to voice, rotating/brightness changing facial images) are used, enabling continuous improvement of recognition accuracy. Application fields include UX optimization for personal genetic testing services, patient stress management in medical institutions, data collection efficiency for insurance companies, and health data collection support for local governments.

The receiving unit can analyze the user's past upload history at the time of uploading genetic test results and select an optimal upload method. The receiving unit analyzes the user's past upload history and selects an optimal upload method. For example, the receiving unit preferentially proposes upload methods (manual, voice input, etc.) that the user has used in the past. The receiving unit selects the most efficient method based on the user's past upload history. The receiving unit automatically selects the method that the user was able to upload without problems in the past. Thus, the receiving unit can provide the optimal upload method for the user. Specifically, the receiving unit accepts as input data a user-specific upload history database (e.g., structured data recording upload date and time, device type used, communication method, UI selection history, number of errors, required time, etc.). As the AI model, a history analysis module combining LSTM or Transformer Encoder for time series analysis and clustering algorithms (e.g., k-means, DBSCAN) can be used. Example inputs include history records such as “2024/06/01 20:15 manual PC success,” “2024/06/10 08:30 voice input smartphone failure,” “2024/06/15 21:00 automatic upload success.” The AI model extracts features (e.g., success rate, average required time, error frequency, user operation tendency) from each history and calculates efficiency scores and reliability scores for each upload method (e.g., manual=0.92, voice=0.65, automatic=0.98, etc.). Example outputs include “recommended method=automatic upload,” “recommended method=voice input,” “recommended method=manual.” Based on these AI outputs, the receiving unit automatically switches the UI of the user terminal (e.g., default display of automatic upload UI, emphasis on voice input button) or optimizes the order of presenting upload method options. Subsequently, the selected upload method is linked to the upload execution module and user notification module, contributing to optimization of user experience and reduction of errors. As a technical effect, this receiving unit, unlike conventional uniform upload method presentation or user-dependent selection, can automatically provide the optimal upload experience according to each user's operation proficiency and environment by AI-based history analysis and dynamic UI optimization, thereby greatly improving upload success rate and user satisfaction. Furthermore, accumulation of history data enables continuous learning of the AI model and improvement of upload method personalization accuracy. Application fields include personal genetic testing services, data linkage support for medical institutions, automated risk assessment for insurance companies, and health data collection platforms for local governments.

The receiving unit can perform filtering at the time of uploading genetic test results based on the user's current health status and lifestyle habits. The receiving unit performs filtering at the time of uploading genetic test results based on the user's current health status and lifestyle habits. For example, if the user's health status is good, the receiving unit provides detailed upload procedures. If the user's health status is poor, the receiving unit provides simplified upload procedures. The receiving unit proposes the optimal upload timing based on the user's lifestyle habits. Thus, the receiving unit can provide appropriate upload procedures according to the user's current health status and lifestyle habits. Specifically, the receiving unit accepts as input data health status questionnaires entered by the user (e.g., numerical vectors or categorical data for physical condition score, sleep duration, stress level, exercise amount, meal frequency, etc.) and lifestyle habit history (e.g., time series data for steps in the past week, drinking/smoking records, medication status, etc.). As the AI model, a multilayer perceptron for health status classification and a time series neural network (e.g., LSTM, GRU) for lifestyle habit pattern extraction can be used. Example inputs include “condition=good, sleep=7.5 h, steps=9000, drinking=0” or “condition=poor, sleep=4.5 h, steps=2000, drinking=2.” The AI model outputs health status labels (e.g., good, poor, caution required) and lifestyle habit clusters (e.g., active type, low activity type, night type, etc.) from these inputs and executes an upload procedure selection algorithm (e.g., detailed procedure for good health, one-tap simplified procedure for poor condition, night notification for night type). Example outputs include “procedure=detailed,” “procedure=simplified,” “timing=night,” etc. Based on these AI outputs, the receiving unit automatically adjusts the upload UI and notification timing of the user terminal to minimize user burden. Subsequently, the selected procedure and timing are linked to the upload execution module and user notification module. As a technical effect, this receiving unit, unlike conventional uniform upload procedures or timing presentation, enables flexible data acquisition tailored to the user's physical condition and lifestyle rhythm by AI-based health status and lifestyle habit analysis and dynamic procedure optimization, thereby greatly improving upload completion rate and user satisfaction. Furthermore, accumulation of health and lifestyle habit data enables continuous learning of the AI model and improvement of procedure optimization accuracy. Application fields include personal genetic testing services, reduction of patient burden in medical institutions, health data collection support for insurance companies, and public health measures for local governments.

The receiving unit can estimate the user's emotions and determine the priority of genetic test results to be uploaded based on the estimated emotions. The receiving unit estimates the user's emotions and determines the priority of genetic test results to be uploaded based on the estimated emotions. For example, if the user is feeling stressed, the receiving unit prioritizes uploading important genetic test results. If the user is relaxed, the receiving unit uploads all genetic test results sequentially. If the user is in a hurry, the receiving unit uploads only the most important genetic test results. Emotion estimation is realized by using an emotion estimation function with, for example, an emotion engine or generative AI. The generative AI may be a text generative AI (e.g., LLM) or a multimodal generative AI, but is not limited to these examples. Thus, the receiving unit can upload genetic test results with the optimal priority according to the user's emotions. Specifically, the receiving unit accepts as input data voice data (e.g., 16 kHz, 16 bit PCM format), text input (e.g., “I'm in a hurry today,” “I'm calm now,” etc.), and facial images (e.g., 224×224 pixel RGB images) from the user terminal for emotion estimation AI. As the emotion estimation AI, a multimodal neural network integrating a Transformer for voice, CNN for facial expression, and a large language model for text can be used. Example inputs include “voice: fast speech,” “text: in a hurry,” “image: frowning face.” The AI model extracts features from each modality and outputs emotion categories (e.g., stress, relaxation, hurry, etc.) and emotion scores (e.g., stress level 0.80, hurry level 0.65, etc.). Example outputs include “emotion=stress,” “emotion=relaxation,” “emotion=hurry,” etc. The receiving unit combines these emotion estimation results with importance information for genetic test results (e.g., priority labels for SNPs directly related to disease risk, lifestyle-related SNPs, etc.) and executes a priority determination algorithm (e.g., only top-priority items during stress, all items during relaxation, only most important items during hurry). Example outputs include “priority list=[rs1234567, rs9876543],” “upload targets=top 5 important items,” etc. Subsequently, the determined priority is linked to the upload execution module and user notification module and reflected in the upload UI and progress display. As a technical effect, this receiving unit, unlike conventional uniform upload order or user-dependent selection, can reduce the user's psychological and time burden and achieve rapid acquisition of important data and improved upload completion rate by AI-based emotion estimation and dynamic priority control. Furthermore, for training emotion estimation AI and priority determination algorithms, cross-entropy loss and utilization of history data are used, enabling continuous improvement of accuracy. Application fields include personal genetic testing services, emergency data collection for medical institutions, automated risk assessment for insurance companies, and health data collection support for local governments.

The receiving unit can consider the user's geographic location information at the time of uploading genetic test results and preferentially upload highly relevant results. The receiving unit considers the user's geographic location information at the time of uploading genetic test results and preferentially uploads highly relevant results. For example, if the user lives in a specific region, the receiving unit prioritizes uploading genetic test results related to that region. If the user is traveling, the receiving unit prioritizes uploading genetic test results related to the current location. If the user is planning to move, the receiving unit prioritizes uploading genetic test results related to the new address. Thus, the receiving unit can upload optimal genetic test results based on the user's geographic location information. Specifically, the receiving unit accepts as input data geographic location information obtained from the user terminal's GPS or IP address (e.g., latitude/longitude pairs, prefecture/city codes, country codes, etc.). In addition, genetic test result data is provided with metadata for region-specific disease risk SNPs and environmental factor-related SNPs (e.g., pollen allergy risk, UV sensitivity, region-specific dietary habit-related genes, etc.). As the AI model, a graph neural network for estimating the relationship between geographic information and genetic information or a rule-based region mapping algorithm can be used. Example inputs include “location: Shinjuku, Tokyo,” “location: Sapporo, Hokkaido,” “location: California, USA,” etc. The AI model calculates region relevance scores between the current or planned location and genetic test results (e.g., pollen allergy risk SNP=high, UV sensitivity SNP=moderate) and outputs a prioritized upload list (e.g., top region-relevant SNP list). Example outputs include “priority upload=[rs1234567, rs2345678],” “region relevance=high,” etc. Based on these AI outputs, the receiving unit automatically determines the target data and order for upload and optimizes the UI and notification content of the user terminal. Subsequently, the prioritized upload data is linked to the analysis unit and determination unit and utilized for region-specific health risk assessment and intervention proposals. As a technical effect, this receiving unit, unlike conventional uniform data upload or region-agnostic data acquisition, can achieve region-specific data acquisition tailored to local health risks and environmental factors by AI-based integration and analysis of geographic and genetic information, greatly improving the accuracy and regional suitability of health management. Furthermore, accumulation of geographic and genetic data enables continuous learning of the AI model and advancement of region-specific services. Application fields include regional optimization of personal genetic testing services, regional epidemiology support for medical institutions, regional risk assessment for insurance companies, and public health measures for local governments.

The receiving unit can analyze the user's social media activity at the time of uploading genetic test results and upload relevant results. The receiving unit analyzes the user's social media activity at the time of uploading genetic test results and uploads relevant results. For example, the receiving unit uploads genetic test results related to health information shared by the user on social media. The receiving unit uploads genetic test results related to information from health experts followed by the user on social media. The receiving unit uploads genetic test results related to information from health communities the user participates in on social media. Thus, the receiving unit can upload optimal genetic test results based on the user's social media activity. Specifically, the receiving unit accepts as input data social media post data obtained within the scope permitted by the user (e.g., health-related post text, list of followed accounts, group participation information in JSON format). As the AI model, an analysis module combining a Transformer-based large language model for natural language processing, a graph neural network for network analysis, and clustering algorithms (e.g., topic modeling, community detection) can be used. Example inputs include “post: I recently started dieting,” “follow: diabetes specialist,” “group: hypertension countermeasure community,” etc. The AI model extracts health interest topics (e.g., diet, diabetes, hypertension, etc.) and keywords from post text, matches them with followed accounts and group participation information, and identifies related disease categories. Example outputs include “related disease=diabetes,” “related disease=hypertension,” “interest topic=diet,” etc. The receiving unit matches these AI outputs with disease relevance metadata of genetic test results (e.g., diabetes risk SNP, hypertension risk SNP, etc.) and extracts highly relevant test results as a prioritized upload list. Example outputs include “priority upload=[rs1234567, rs2345678],” etc. Subsequently, the prioritized upload data is linked to the analysis unit and determination unit and utilized for risk assessment and intervention proposals tailored to the user's health interests. As a technical effect, this receiving unit, unlike conventional uniform data upload or acquisition ignoring user interests, can achieve data acquisition tailored to the user's latest health interests and behavioral changes by AI-based analysis of social media activity and relevance estimation, greatly improving the personalization accuracy and user satisfaction of health management. Furthermore, accumulation of social media data enables continuous learning of the AI model and tracking of interest trends. Application fields include enhancement of personalization in personal genetic testing services, patient interest analysis for medical institutions, advanced risk assessment for insurance companies, and health promotion measures for local governments.

The analysis unit can estimate the user's emotions and adjust the method of presenting analysis based on the estimated emotions. The analysis unit estimates the user's emotions and adjusts the method of presenting analysis based on the estimated emotions. For example, if the user is relaxed, the analysis unit provides detailed analysis results. If the user is stressed, the analysis unit provides concise analysis results. If the user is excited, the analysis unit provides visually appealing analysis results. Emotion estimation is realized by using an emotion estimation function with, for example, an emotion engine or generative AI. The generative AI may be a text generative AI (e.g., LLM) or a multimodal generative AI, but is not limited to these examples. Thus, the analysis unit can provide analysis results in the optimal presentation method according to the user's emotions. Specifically, the analysis unit accepts as input data voice data obtained from the user terminal (e.g., 16 kHz, 16 bit PCM format audio waveform), text input (e.g., natural language sentences such as “I'm calm today,” “I'm busy now”), or facial images (e.g., 224×224 pixel RGB images) for emotion estimation AI. The analysis unit can use a multimodal neural network combining a Transformer-based model for voice recognition, CNN for facial expression recognition, and a large language model for text emotion classification as the emotion estimation AI. The analysis unit extracts acoustic features (e.g., MFCC, pitch, energy), text embedding vectors (e.g., 768-dimensional BERT-based vectors), and image feature maps (e.g., 512-dimensional ResNet output vectors), and outputs emotion categories (e.g., stress, relaxation, excitement, etc.) and emotion scores (e.g., stress level 0.82, relaxation level 0.15, etc.) in the integration layer. Example inputs include “voice: slow speech,” “text: I'm calm today,” “image: smiling.” Based on the output of the emotion estimation AI, the analysis unit dynamically switches the method of presenting analysis results. For example, if relaxation level is high, a detailed analysis report including numerical data, graphs, and explanations of causal relationships is generated; if stress level is high, only key points are presented in bullet points; if excitement level is high, interactive visual analysis using colors and animations is provided. When determining the method of presenting analysis results, the analysis unit also considers the user's past browsing history and preferences to realize a personalized UI/UX. The analysis unit automatically generates output formats such as text summaries, detailed reports, infographics, and dashboards. Example outputs include “Type 2 diabetes risk=0.78, main factors: rs1234567 (T/T), high-fat diet (with detailed explanation)” or “Hypertension risk=0.45 (key points only).” Subsequently, the output of the analysis unit is linked to the visualization unit and proposal unit and utilized for feedback and intervention proposals optimized for the user's emotional state. As a technical effect, the analysis unit, unlike conventional uniform presentation of analysis results or manual adjustment of presentation, can automatically generate analysis results in real time tailored to the user's psychological state and situation by AI-based multimodal emotion estimation and dynamic presentation optimization, greatly improving user understanding, acceptance, and behavioral change rate. Furthermore, for emotion estimation AI training, cross-entropy loss and data augmentation (e.g., adding noise to voice, rotating/brightness changing facial images) are used, enabling continuous improvement of recognition accuracy. Application fields include UX optimization for personal health management services, patient explanation support in medical institutions, risk assessment reports for insurance companies, and public health monitoring for local governments.

The analysis unit can adjust the level of detail of analysis based on the importance of genetic information during analysis. The analysis unit adjusts the level of detail of analysis based on the importance of genetic information during analysis. For example, the analysis unit provides detailed analysis results for important genetic information. For less important genetic information, the analysis unit provides concise analysis results. The analysis unit prioritizes analysis of highly important genetic information based on user interest. Thus, the analysis unit can analyze important information in detail and provide it to the user. Specifically, the analysis unit accepts as input data genetic test data (e.g., SNP sequence vectors for 22,000 genetic loci, 0/1/2 representation for each rsID). The analysis unit assigns importance scores (e.g., evidence scores based on literature, AI-estimated contribution values) to each genetic locus or SNP for disease risk and lifestyle relevance. For genetic information with high importance scores, the analysis unit performs detailed statistical analysis (e.g., odds ratio, risk contribution, interaction analysis, etc.) and visualization (e.g., heat maps, detailed graphs), and for genetic information with low importance scores, outputs only summary values or simple labels. The analysis unit extracts highly important genetic information as analysis targets based on user interest areas (e.g., diabetes, hypertension, obesity, etc.) and past browsing history. As the AI model, a multilayer perceptron for estimating the importance of genetic information and a statistical model for estimating disease risk contribution (e.g., logistic regression, decision tree) can be used in combination. Example inputs include “rs1234567=2 (importance=0.92),” “rs9876543=1 (importance=0.15),” etc. The analysis unit automatically generates a detailed analysis target list and a simple analysis target list according to importance scores, and example outputs include “detailed analysis: rs1234567 (contribution 0.12, with detailed explanation), simple analysis: rs9876543 (summary only).” Subsequently, detailed analysis results are linked to the determination unit and proposal unit and used as supporting data for individualized health management proposals and risk assessment. As a technical effect, the analysis unit, unlike conventional uniform genetic information analysis or manual importance determination, can efficiently and accurately extract and analyze truly important information for the user from vast genetic data by AI-based importance scoring and dynamic detail control, greatly improving the usefulness of health management and user satisfaction. Furthermore, accumulation of importance scores and analysis history enables continuous learning of the AI model and improvement of analysis accuracy. Application fields include automatic report generation for personal genetic testing services, patient explanation support in medical institutions, risk assessment for insurance companies, and public health measures for local governments.

The analysis unit can apply different analysis algorithms according to the category of genetic information during analysis. The analysis unit applies different analysis algorithms according to the category of genetic information during analysis. For example, the analysis unit applies specific analysis algorithms to genetic information related to health risk. The analysis unit applies different analysis algorithms to genetic information related to physical ability. The analysis unit applies yet another analysis algorithm to genetic information related to dietary habits. Thus, the analysis unit can provide appropriate analysis results according to the category of genetic information. Specifically, the analysis unit accepts as input data genetic test data (e.g., SNP sequence vectors for 22,000 genetic loci, 0/1/2 representation for each rsID) and category labels assigned to each genetic information (e.g., health risk, physical ability, dietary habits, etc.). The analysis unit automatically selects different analysis algorithms for each category. For example, for the health risk category, a multilayer perceptron or logistic regression model for disease risk estimation is applied; for the physical ability category, pattern matching or clustering algorithms for muscle strength/endurance-related genes are applied; and for the dietary habits category, network analysis or recommendation algorithms for ingredient preferences and metabolism-related genes are applied. Example inputs include “rs1234567=2 (category=health risk),” “rs2345678=1 (category=physical ability),” “rs3456789=0 (category=dietary habits),” etc. The analysis unit integrates analysis results for each category, and example outputs include “type 2 diabetes risk=0.78 (health risk algorithm),” “muscle strength type=high (physical ability algorithm),” “lipid metabolism type=low (dietary habits algorithm),” etc. Subsequently, category-specific analysis results are linked to the determination unit and proposal unit and used as supporting data for disease risk assessment and individualized proposals for diet and exercise. As a technical effect, the analysis unit, unlike conventional uniform genetic information analysis or manual category determination, can achieve high-precision and efficient analysis tailored to the diverse characteristics of genetic information by AI-based automatic category determination and algorithm optimization, greatly improving the accuracy of health management and user satisfaction. Furthermore, accumulation of category-specific analysis history and algorithm selection data enables continuous learning of the AI model and improvement of analysis accuracy. Application fields include multipurpose report generation for personal genetic testing services, patient explanation support in medical institutions, ability assessment in sports fields, and risk assessment for insurance companies.

The analysis unit can estimate the user's emotions and adjust the length of analysis based on the estimated emotions. The analysis unit estimates the user's emotions and adjusts the length of analysis based on the estimated emotions. For example, if the user is in a hurry, the analysis unit provides a short, concise analysis result. If the user is relaxed, the analysis unit provides a detailed analysis result. If the user is excited, the analysis unit provides a visually appealing analysis result. Emotion estimation is realized by using an emotion estimation function with, for example, an emotion engine or generative AI. The generative AI may be a text generative AI (e.g., LLM) or a multimodal generative AI, but is not limited to these examples. Thus, the analysis unit can provide analysis results with the optimal length according to the user's emotions. Specifically, the analysis unit accepts as input data voice data (e.g., 16 kHz, 16 bit PCM format), text input (e.g., “I'm in a hurry today,” “I'm calm now,” etc.), and facial images (e.g., 224×224 pixel RGB images) from the user terminal for emotion estimation AI. The analysis unit can use a multimodal neural network combining a Transformer-based model for voice recognition, CNN for facial expression recognition, and a large language model for text emotion classification. The analysis unit extracts acoustic features, text embedding vectors, and image feature maps, and outputs emotion categories (e.g., stress, relaxation, hurry, etc.) and emotion scores (e.g., stress level 0.80, hurry level 0.65, etc.) in the integration layer. Example inputs include “voice: fast speech,” “text: in a hurry,” “image: frowning face.” Based on the output of the emotion estimation AI, the analysis unit dynamically adjusts the length of analysis results. For example, if hurry level is high, only key points are presented in bullet points; if relaxation level is high, a detailed analysis report including numerical data, graphs, and explanations of causal relationships is generated; if excitement level is high, interactive visual analysis using colors and animations is provided. When determining the length and presentation method of analysis results, the analysis unit also considers the user's past browsing history and preferences to realize a personalized UI/UX. Example outputs include “type 2 diabetes risk=0.78 (key points only),” “hypertension risk=0.45 (with detailed explanation),” etc. Subsequently, the output of the analysis unit is linked to the visualization unit and proposal unit and utilized for feedback and intervention proposals optimized for the user's emotional state. As a technical effect, the analysis unit, unlike conventional uniform presentation of analysis results or manual adjustment of length, can automatically generate analysis results in real time tailored to the user's psychological state and situation by AI-based multimodal emotion estimation and dynamic length optimization, greatly improving user understanding, acceptance, and behavioral change rate. Furthermore, for emotion estimation AI training, cross-entropy loss and data augmentation are used, enabling continuous improvement of recognition accuracy. Application fields include UX optimization for personal health management services, patient explanation support in medical institutions, risk assessment reports for insurance companies, and public health monitoring for local governments.

The analysis unit can determine the priority of analysis based on the submission timing of genetic information during analysis. The analysis unit determines the priority of analysis based on the submission timing of genetic information during analysis. For example, the analysis unit prioritizes analysis of recently submitted genetic information. The analysis unit postpones analysis of older genetic information. The analysis unit adjusts the analysis schedule based on submission timing. Thus, the analysis unit can prioritize analysis of the latest information and provide it to the user. Specifically, the analysis unit accepts as input data genetic data with metadata including submission date and time or timestamp information (e.g., ISO8601 format date and time, UNIX timestamp, etc.). The analysis unit automatically calculates priority scores for each submission timing, assigns higher priority to newer data, and lower priority to older data. Based on priority scores, the analysis unit uses a scheduling algorithm for analysis jobs (e.g., priority queue, round robin, FIFO, etc.) to automatically determine the order of analysis. Example inputs include “submission date=2024-06-20 10:00,” “submission date=2024-05-15 09:30,” etc. The analysis unit prioritizes analysis of newer data, and example outputs include “priority analysis: 2024-06-20 data,” “postponed: 2024-05-15 data,” etc. Subsequently, prioritized analysis results are immediately linked to the determination unit and proposal unit and utilized for rapid feedback and intervention proposals to the user. As a technical effect, the analysis unit, unlike conventional uniform analysis order or manual scheduling, can achieve rapid analysis of the latest data and timely information provision to the user by AI-based submission timing analysis and dynamic priority control, greatly improving the real-time nature of health management and user satisfaction. Furthermore, accumulation of submission timing data enables continuous learning of the AI model and improvement of scheduling accuracy. Application fields include real-time report generation for personal genetic testing services, emergency data analysis for medical institutions, automated risk assessment for insurance companies, and health data collection platforms for local governments.

The analysis unit can adjust the order of analysis based on the relevance of genetic information during analysis. The analysis unit adjusts the order of analysis based on the relevance of genetic information during analysis. For example, the analysis unit prioritizes the analysis of highly relevant genetic information. The analysis unit postpones the analysis of genetic information with low relevance. The analysis unit adjusts the analysis schedule based on the relevance of genetic information. As a result, the analysis unit can prioritize and provide highly relevant information to the user. Specifically, the analysis unit receives genetic test data (e.g., SNP sequence vector of 22,000 loci) and relevance scores for each genetic information (e.g., disease risk contribution, lifestyle relevance, network centrality index, etc.) as input data. The analysis unit quantifies the relevance between genetic information using graph neural networks for relevance estimation or clustering algorithms (e.g., community detection, topic modeling). Examples of input include “rs1234567=2 (relevance=0.91)”, “rs9876543=1 (relevance=0.12)”, etc. The analysis unit adds genetic information with high relevance scores to the analysis target list with priority, while information with low relevance is postponed. The analysis unit executes an analysis order determination algorithm (e.g., descending sort by relevance score, priority queue) to automate the scheduling of analysis jobs. Examples of output include “Priority analysis: rs1234567”, “Postponed: rs9876543”, etc. In subsequent processing, the results of prioritized analysis are linked to the determination unit and proposal unit and used as evidence data for disease risk assessment and individually optimized intervention proposals. As a technical effect, unlike conventional uniform analysis order or manual relevance judgment, the analysis unit can efficiently and accurately extract and analyze truly important information for the user from vast genetic data by AI-based relevance estimation and dynamic order optimization, thereby greatly improving the usefulness of health management and user satisfaction. Furthermore, by accumulating relevance scores and analysis history, continuous learning of the AI model and improvement of analysis accuracy are possible. Application fields include automatic report generation for personal genetic testing services, patient explanation support in medical institutions, risk assessment for insurance companies, and public health measures for local governments.

The determination unit can estimate the user's emotions and adjust the criteria for determining disease risk based on the estimated emotions. The determination unit estimates the user's emotions and adjusts the criteria for determining disease risk based on the estimated emotions. For example, the determination unit applies detailed criteria when the user is relaxed. The determination unit applies concise criteria when the user feels stressed. The determination unit applies visually appealing criteria when the user is excited. Emotion estimation is realized by using emotion estimation functions, such as emotion engines or generative AI. Generative AI includes text generation AI (e.g., LLM) and multimodal generative AI, but is not limited to these examples. As a result, the determination unit can determine disease risk using optimal criteria according to the user's emotions. Specifically, the determination unit receives genetic test data from the analysis unit (e.g., SNP sequence vector of 22,000 loci, 0/1/2 representation for each rsID), lifestyle vectors (e.g., sleep duration, step count, drinking/smoking habits, etc.), and output from emotion estimation AI (e.g., stress level 0.82, relaxation level 0.15, excitement level 0.03, etc.) as input data. The determination unit can use a multimodal neural network combining a Transformer-based model for speech recognition, a CNN for facial expression recognition, and a large language model for text emotion classification as the emotion estimation AI. Examples of input include “Speech: slow utterance”, “Text: calm today”, “Image: smile”, etc. The emotion estimation AI extracts acoustic features (e.g., MFCC, pitch, energy), text embedding vectors (e.g., BERT-based 768-dimensional vector), and image feature maps (e.g., ResNet output 512-dimensional vector), and outputs emotion categories and emotion scores in the integration layer. The determination unit dynamically switches parameters and thresholds of the disease risk determination algorithm according to the emotion scores. For example, when the relaxation level is high, detailed statistical models (e.g., multivariate logistic regression, Bayesian estimation, risk contribution decomposition, etc.) are applied to calculate risk scores for each disease, and the contribution and causal relationships of explanatory variables are analyzed in detail. When the stress level is high, only major risk factors are extracted and a simple rule-based determination (e.g., risk score≥0.7 is high risk, <0.3 is low risk, etc.) is applied. When the excitement level is high, the determination results are presented in an interactive visual format using colors and animations. When switching criteria, the determination unit also considers the user's past determination history and preferences to realize a personalized determination experience. The output of the determination unit is structured as disease-specific risk labels (e.g., “Type 2 diabetes risk=high”, “Hypertension risk=medium”), risk scores (e.g., 0.78, 0.45, etc.), and determination rationale (e.g., rs 1234567 (T/T), high-fat diet, sleep deprivation, etc.). Examples of output include “Type 2 diabetes risk=0.78 (with detailed explanation)”, “Hypertension risk=0.45 (summary only)”, etc. In subsequent processing, the output of the determination unit is linked to the proposal unit and visualization unit and used for intervention proposals and feedback generation optimized for the user's emotional state. As a technical effect, unlike conventional uniform criteria or manual criteria adjustment, the determination unit can automatically generate determination results tailored to the user's psychological state and situation in real time by AI-based multimodal emotion estimation and dynamic criteria optimization, greatly improving user understanding, acceptance, and behavior change rates. Furthermore, cross-entropy loss and data augmentation (e.g., adding noise to speech, rotating/changing brightness of facial images) are used for continuous improvement of recognition and determination accuracy in emotion estimation AI and determination algorithms. Application fields include UX optimization for personal health management services, patient explanation support in medical institutions, risk assessment reports for insurance companies, and public health monitoring for local governments.

The determination unit can improve the accuracy of determination by considering the interrelationships of genetic information during determination. The determination unit improves the accuracy of determination by considering the interrelationships of genetic information during determination. For example, the determination unit analyzes the interrelationships of genetic information to accurately determine disease risk. The determination unit identifies high-risk genes based on the interrelationships of genetic information. The determination unit excludes low-risk genes by considering the interrelationships of genetic information. As a result, the determination unit can improve the accuracy of disease risk determination by considering the interrelationships of genetic information. Specifically, the determination unit receives genetic test data from the analysis unit (e.g., SNP sequence vector of 22,000 loci, 0/1/2 representation for each rsID) as input data. The determination unit can use graph neural networks (GNN), Bayesian networks, or correlation matrix analysis algorithms to estimate interrelationships between genes. Examples of input include SNP vectors such as “rs1234567=2, rs2345678=1, rs3456789=0”. The determination unit constructs a graph structure with these genetic information as nodes and known biological pathways or disease-related network information as edges. The GNN propagates and aggregates feature quantities of each node (e.g., allele type, contribution, disease relevance score, etc.) and learns higher-order interactions between nodes (e.g., epistasis, pathway dependency). The determination unit obtains risk scores for each disease (e.g., type 2 diabetes risk=0.81, dyslipidemia risk=0.39, etc.) and contribution vectors for each risk factor (e.g., rs1234567=0.12, rs2345678=0.09, etc.) as output from the GNN. The determination unit prioritizes gene groups with high interrelationship scores as risk determination targets and excludes genes with weak interactions or low risk contribution from determination. Examples of output include “Risk determination targets: rs1234567, rs2345678 (strong interaction)”, “Excluded: rs3456789 (low contribution)”, etc. In subsequent processing, the determination results are linked to the proposal unit and visualization unit and used as evidence data for individually optimized intervention proposals and risk explanations. As a technical effect, unlike conventional simple single-gene evaluation or manual interrelationship determination, the determination unit can efficiently and accurately extract and determine factors that essentially contribute to disease risk from complex genetic networks by AI-based high-dimensional graph structure analysis and dynamic determination optimization, greatly improving the usefulness of health management and determination accuracy. Furthermore, by accumulating interrelationship scores and determination history, continuous learning of the AI model and improvement of determination accuracy are possible. Application fields include automated risk assessment for personal genetic testing services, patient explanation support in medical institutions, risk assessment for insurance companies, and public health measures for local governments.

The determination unit can perform determination by considering attribute information of the submitter of genetic information during determination. The determination unit performs determination by considering attribute information of the submitter of genetic information during determination. For example, the determination unit determines disease risk based on the submitter's age. The determination unit determines disease risk based on the submitter's gender. The determination unit determines disease risk based on the submitter's lifestyle habits. As a result, the determination unit can perform more accurate disease risk determination by considering the submitter's attribute information. Specifically, the determination unit receives genetic test data from the analysis unit (e.g., SNP sequence vector of 22,000 loci) and user attribute information (e.g., age, gender, family history, lifestyle vector (sleep duration, step count, drinking/smoking habits, etc.)) as input data. The determination unit integrates these attribute information into a multivariate logistic regression model, decision tree algorithm, or deep neural network using attribute embeddings to perform determination processing. Examples of input include “Age=45, Gender=Female, rs1234567=2, Step count=8000, Drinking=1”, etc. The determination unit applies weighting for each attribute, for example, increasing the risk contribution for certain diseases when age is high, reflecting differences in disease susceptibility by gender, and incorporating lifestyle factors (e.g., lack of exercise, smoking habits, etc.) into risk score calculation. The determination unit also considers interaction effects between attribute information and genetic information (e.g., specific genotype×old age×smoking increases risk) to construct complex risk models. Examples of output include “Type 2 diabetes risk=0.78 (reflecting age and lifestyle habits)”, “Hypertension risk=0.45 (reflecting gender and family history)”, etc. In subsequent processing, the determination results are linked to the proposal unit and visualization unit and used as evidence data for individually optimized intervention proposals and risk explanations. As a technical effect, unlike conventional uniform determination or manual consideration of attributes, the determination unit can automatically generate high-precision risk assessments tailored to each user's attributes in real time by AI-based multivariate analysis and dynamic determination optimization, greatly improving the usefulness of health management and user satisfaction. Furthermore, by accumulating attribute data and determination history, continuous learning of the AI model and improvement of determination accuracy are possible. Application fields include enhancement of personalization for personal genetic testing services, patient explanation support in medical institutions, risk assessment for insurance companies, and public health measures for local governments.

The determination unit can estimate the user's emotions and adjust the display order of determination results based on the estimated emotions. The determination unit estimates the user's emotions and adjusts the display order of determination results based on the estimated emotions. For example, the determination unit prioritizes the display of detailed determination results when the user is relaxed. The determination unit prioritizes the display of concise determination results when the user feels stressed. The determination unit prioritizes the display of visually appealing determination results when the user is excited. Emotion estimation is realized by using emotion estimation functions, such as emotion engines or generative AI. Generative AI includes text generation AI (e.g., LLM) and multimodal generative AI, but is not limited to these examples. As a result, the determination unit can display determination results in the optimal order according to the user's emotions. Specifically, the determination unit receives determination result data from the analysis unit (e.g., disease-specific risk scores, risk factor labels, detailed explanation text, etc.) and output from emotion estimation AI (e.g., stress level 0.80, relaxation level 0.15, excitement level 0.05, etc.) as input data. The determination unit can use a multimodal neural network combining a Transformer-based model for speech recognition, a CNN for facial expression recognition, and a large language model for text emotion classification as the emotion estimation AI. Examples of input include “Speech: fast”, “Text: in a hurry”, “Image: frown”, etc. The determination unit dynamically switches the display order determination algorithm for determination results (e.g., descending order by detail score, summary priority, visual priority, etc.) according to the emotion scores. For example, when the relaxation level is high, detailed determination results (e.g., risk scores, causal relationship explanations, graphs, etc.) are displayed at the top; when the stress level is high, only the main points are displayed in bullet points with priority; and when the excitement level is high, interactive determination results using colors and animations are displayed at the top. The determination unit also considers the user's past browsing history and preferences to realize a personalized display order. Examples of output include “Display order: detail→summary→visual”, “Display order: summary→detail→visual”, etc. In subsequent processing, the display order is linked to the visualization unit and user interface and used for feedback and intervention proposals optimized for the user's emotional state. As a technical effect, unlike conventional uniform display order or manual order adjustment, the determination unit can automatically generate determination result presentations tailored to the user's psychological state and situation in real time by AI-based multimodal emotion estimation and dynamic display order optimization, greatly improving user understanding, acceptance, and behavior change rates. Furthermore, cross-entropy loss and data augmentation are used for continuous improvement of recognition accuracy and UX in emotion estimation AI and display order determination algorithms. Application fields include UX optimization for personal health management services, patient explanation support in medical institutions, risk assessment reports for insurance companies, and public health monitoring for local governments.

The determination unit can perform determination by considering the geographic distribution of genetic information during determination. The determination unit performs determination by considering the geographic distribution of genetic information during determination. For example, the determination unit determines disease risk based on genetic information related to a specific region. The determination unit identifies high-risk regions based on geographic distribution. The determination unit excludes low-risk regions by considering geographic distribution. As a result, the determination unit can determine disease risk related to regions. Specifically, the determination unit receives genetic test data from the analysis unit (e.g., SNP sequence vector of 22,000 loci) and the user's geographic location information (e.g., latitude/longitude pair, prefecture/municipality code, country code, etc.) as input data. The determination unit can use graph neural networks for estimating the relevance between genetic information and geographic information or rule-based regional mapping algorithms. Examples of input include “Location=Shinjuku, Tokyo”, “Location=Sapporo, Hokkaido”, “Location=California, USA”, etc. The determination unit utilizes metadata for region-specific disease risk SNPs and environmental factor-related SNPs (e.g., pollen allergy risk, UV sensitivity, region-specific dietary habit-related genes, etc.) to calculate region-relatedness scores between the current or planned location and genetic test results (e.g., pollen allergy risk SNP=high, UV sensitivity SNP=medium, etc.). The determination unit prioritizes genetic information with high region-relatedness as risk determination targets and excludes regions or genetic information with low relatedness from determination. Examples of output include “Risk determination target: rs1234567 (high region-relatedness)”, “Excluded: rs2345678 (low region-relatedness)”, etc. In subsequent processing, the determination results are linked to the proposal unit and visualization unit and used for region-specific health risk assessment and intervention proposals. As a technical effect, unlike conventional uniform determination or risk assessment ignoring regions, the determination unit can realize high-precision determination tailored to region-specific health risks and environmental factors by AI-based integrated analysis of geographic and genetic information, greatly improving the regional suitability of health management and user satisfaction. Furthermore, by accumulating geographic information and genetic data, continuous learning of the AI model and advancement of region-specific services are possible. Application fields include regional optimization for personal genetic testing services, regional epidemiology support in medical institutions, regional risk assessment for insurance companies, and public health measures for local governments.

The determination unit can improve the accuracy of determination by referring to related literature of genetic information during determination. The determination unit improves the accuracy of determination by referring to related literature of genetic information during determination. For example, the determination unit refers to related literature to accurately determine disease risk. The determination unit identifies high-risk genes based on related literature. The determination unit excludes low-risk genes by referring to related literature. As a result, the determination unit can improve the accuracy of disease risk determination by referring to related literature. Specifically, the determination unit receives genetic test data from the analysis unit (e.g., SNP sequence vector of 22,000 loci) and a related literature database linked to each genetic information (e.g., PubMed ID, evidence level, paper summary text, etc.) as input data. The determination unit can use large language models for natural language processing (e.g., Transformer-based) or literature evidence scoring algorithms to evaluate the relationship between each genetic SNP and disease risk based on literature. Examples of input include “rs1234567=2 (PubMed: 12345678, evidence=high)”, “rs2345678=1 (PubMed: 23456789, evidence=low)”, etc. The determination unit extracts disease risk-related keywords and evidence scores from literature summary texts and reflects them in the risk determination algorithm. The determination unit prioritizes genetic information with high evidence scores as risk determination targets and excludes genetic information with insufficient evidence from determination. Examples of output include “Risk determination target: rs1234567 (high evidence)”, “Excluded: rs2345678 (low evidence)”, etc. In subsequent processing, the determination results are linked to the proposal unit and visualization unit and used for evidence-based risk assessment and intervention proposals. As a technical effect, unlike conventional uniform determination or manual literature reference, the determination unit can automatically generate high-precision risk assessments based on scientific evidence in real time by AI-based automatic literature analysis and evidence scoring, greatly improving the reliability of health management and user acceptance. Furthermore, by accumulating literature data and determination history, continuous learning of the AI model and improvement of determination accuracy are possible. Application fields include evidence-based report generation for personal genetic testing services, patient explanation support in medical institutions, risk assessment for insurance companies, and public health measures for local governments.

The proposal unit can estimate the user's emotions and adjust the method of presenting proposals based on the estimated emotions. The proposal unit estimates the user's emotions and adjusts the method of presenting proposals based on the estimated emotions. For example, the proposal unit provides detailed proposals when the user is relaxed. The proposal unit provides concise proposals when the user feels stressed. The proposal unit provides visually appealing proposals when the user is excited. Emotion estimation is realized by using emotion estimation functions, such as emotion engines or generative AI. Generative AI includes text generation AI (e.g., LLM) and multimodal generative AI, but is not limited to these examples. As a result, the proposal unit can provide proposals using the optimal presentation method according to the user's emotions. Specifically, the proposal unit receives voice data obtained from the user terminal (e.g., 16 kHz, 16 bit PCM waveform), text input (e.g., “I am calm today”, “I am busy now”, etc.), and facial images (e.g., 224×224 pixel RGB images) as input data for emotion estimation AI. The proposal unit can use a multimodal neural network combining a Transformer-based model for speech recognition, a CNN for facial expression recognition, and a large language model for text emotion classification as the emotion estimation AI. The proposal unit extracts acoustic features (e.g., MFCC, pitch, energy), text embedding vectors (e.g., BERT-based 768-dimensional vector), and image feature maps (e.g., ResNet output 512-dimensional vector), and outputs emotion categories (e.g., stress, relaxation, excitement, etc.) and emotion scores (e.g., stress level 0.82, relaxation level 0.15, etc.) in the integration layer. Examples of input include “Speech: slow utterance”, “Text: calm today”, “Image: smile”, etc. Based on the output of the emotion estimation AI, the proposal unit dynamically switches the method of presenting proposal content. For example, when the relaxation level is high, a proposal report including detailed nutritional information, cooking procedures, ingredient origin information, and scientific rationale is generated; when the stress level is high, only the main points are presented in bullet points; and when the excitement level is high, interactive visual proposals using colors and animations are provided. When determining the method of presenting proposal content, the proposal unit also considers the user's past browsing history and preferences to realize a personalized UI/UX. The proposal unit automatically generates output formats for proposal results, such as text summaries, detailed reports, infographics, dashboards, etc. Examples of output include “Low-carb Japanese-style menu with chicken and tomato as main ingredients (with detailed explanation)”, “Gluten-free meal kit without wheat (main points only)”, etc. In subsequent processing, the output of the proposal unit is linked to the delivery scheduler and user notification module and used for feedback and intervention proposals optimized for the user's emotional state. As a technical effect, unlike conventional uniform proposal content presentation or manual adjustment of presentation, the proposal unit can automatically generate proposal content tailored to the user's psychological state and situation in real time by AI-based multimodal emotion estimation and dynamic presentation optimization, greatly improving user understanding, acceptance, and behavior change rates. Furthermore, cross-entropy loss and data augmentation (e.g., adding noise to speech, rotating/changing brightness of facial images) are used for continuous improvement of recognition accuracy in emotion estimation AI. Application fields include UX optimization for personal health management services, patient explanation support in medical institutions, risk assessment reports for insurance companies, and public health monitoring for local governments.

The proposal unit can adjust the level of detail of proposals based on the importance of the meal kit during proposal. The proposal unit adjusts the level of detail of proposals based on the importance of the meal kit during proposal. For example, the proposal unit provides detailed proposals for important meal kits. The proposal unit provides concise proposals for meal kits with low importance. The proposal unit prioritizes proposals for meal kits with high importance based on the user's interests. As a result, the proposal unit can provide detailed proposals for important information to the user. Specifically, the proposal unit receives importance scores assigned to each meal kit (e.g., contribution to disease risk reduction, nutritional balance score, degree of match with user's health goals, etc.) and the user's areas of interest (e.g., low-carb, allergy-friendly, muscle enhancement, etc.) as input data. The proposal unit inputs these data into a multilayer perceptron, decision tree algorithm, or importance scoring AI model to automatically determine the level of detail for each meal kit (e.g., detailed, standard, simple). Examples of input include “Meal kit A (importance=0.92, interest=high)”, “Meal kit B (importance=0.15, interest=low)”, etc. For meal kits with high importance, the proposal unit generates a proposal report including detailed nutritional information, cooking procedures, scientific rationale, ingredient origin information, and explanations of disease risk reduction effects; for meal kits with low importance, only the main points are presented in bullet points. Furthermore, the proposal unit considers the user's past selection history and satisfaction scores to prioritize detailed proposals for areas of high interest. Examples of output include “Detailed proposal: low-carb Japanese-style menu (with detailed explanation)”, “Simple proposal: gluten-free meal kit (main points only)”, etc. In subsequent processing, the proposal content is linked to the delivery scheduler and user notification module, contributing to optimization of user experience and improvement of satisfaction. As a technical effect, unlike conventional uniform proposal detail or manual importance judgment, the proposal unit can efficiently and accurately extract and propose truly important information for the user by AI-based importance scoring and dynamic detail control, greatly improving the usefulness of health management and user satisfaction. Furthermore, by accumulating importance scores and proposal history, continuous learning of the AI model and improvement of proposal accuracy are possible. Application fields include personal health food subscription services, meal proposals for patients in medical institutions, health intervention programs for insurance companies, and health promotion measures for local governments.

The proposal unit can apply different proposal algorithms according to the category of the meal kit during proposal. The proposal unit applies different proposal algorithms according to the category of the meal kit during proposal. For example, the proposal unit applies a specific proposal algorithm to meal kits related to health risks. The proposal unit applies another proposal algorithm to meal kits related to physical performance. The proposal unit applies yet another proposal algorithm to meal kits related to dietary habits. As a result, the proposal unit can provide appropriate proposals according to the category of the meal kit. Specifically, the proposal unit receives category labels assigned to each meal kit (e.g., health risk reduction, physical performance improvement, dietary habit improvement, etc.) and the user's health goals and lifestyle data as input data. The proposal unit automatically selects different proposal algorithms for each category. For example, for the health risk category, linear programming or multivariate optimization algorithms are applied to maximize disease risk reduction effects; for the physical performance category, ingredient combination search or pattern matching algorithms are applied to enhance muscle strength and endurance; and for the dietary habit category, recommendation systems or network analysis algorithms reflecting preferences and allergy constraints are applied. Examples of input include “Meal kit A (category=health risk)”, “Meal kit B (category=physical performance)”, “Meal kit C (category=dietary habit)”, etc. The proposal unit integrates proposal results for each category, and examples of output include “Low-carb Japanese-style menu (health risk algorithm)”, “High-protein muscle enhancement menu (physical performance algorithm)”, “Gluten-free meal kit (dietary habit algorithm)”, etc. In subsequent processing, category-specific proposal results are linked to the delivery scheduler and user notification module and used as evidence data for individually optimized meal proposals and health intervention plans. As a technical effect, unlike conventional uniform proposal algorithms or manual category judgment, the proposal unit can realize high-precision and efficient proposals tailored to the diverse characteristics of meal kits by AI-based automatic category determination and algorithm optimization, greatly improving the accuracy of health management and user satisfaction. Furthermore, by accumulating proposal history and algorithm selection data for each category, continuous learning of the AI model and improvement of proposal accuracy are possible. Application fields include multipurpose proposals for personal health food services, dietary guidance for patients in medical institutions, performance enhancement meal proposals in sports fields, and health intervention programs for insurance companies.

The proposal unit can adjust the length of proposals based on the user's emotions. The proposal unit estimates the user's emotions and adjusts the length of proposals based on the estimated emotions. For example, the proposal unit provides short, concise proposals when the user is in a hurry. The proposal unit provides detailed proposals when the user is relaxed. The proposal unit provides visually appealing proposals when the user is excited. Emotion estimation is realized by using emotion estimation functions, such as emotion engines or generative AI. Generative AI includes text generation AI (e.g., LLM) and multimodal generative AI, but is not limited to these examples. As a result, the proposal unit can provide proposals with optimal length according to the user's emotions. Specifically, the proposal unit receives voice data obtained from the user terminal (e.g., 16 kHz, 16 bit PCM format), text input (e.g., “I am in a hurry today”, “I am calm now”, etc.), and facial images (e.g., 224×224 pixel RGB images) as input data for emotion estimation AI. The proposal unit can use a multimodal neural network combining a Transformer-based model for speech recognition, a CNN for facial expression recognition, and a large language model for text emotion classification. The proposal unit extracts acoustic features, text embedding vectors, and image feature maps, and outputs emotion categories (e.g., stress, relaxation, hurry, etc.) and emotion scores (e.g., stress level 0.80, hurry level 0.65, etc.) in the integration layer. Examples of input include “Speech: fast”, “Text: in a hurry”, “Image: frown”, etc. Based on the output of the emotion estimation AI, the proposal unit dynamically adjusts the length of proposal content. For example, when the hurry level is high, only the main points are presented in bullet points; when the relaxation level is high, a proposal report including detailed nutritional information, cooking procedures, and scientific rationale is generated; and when the excitement level is high, interactive visual proposals using colors and animations are provided. When determining the length and presentation method of proposal content, the proposal unit also considers the user's past browsing history and preferences to realize a personalized UI/UX. Examples of output include “Low-carb Japanese-style menu (main points only)”, “Gluten-free meal kit (with detailed explanation)”, etc. In subsequent processing, the output of the proposal unit is linked to the delivery scheduler and user notification module and used for feedback and intervention proposals optimized for the user's emotional state. As a technical effect, unlike conventional uniform proposal content presentation or manual length adjustment, the proposal unit can automatically generate proposal content tailored to the user's psychological state and situation in real time by AI-based multimodal emotion estimation and dynamic length optimization, greatly improving user understanding, acceptance, and behavior change rates. Furthermore, cross-entropy loss and data augmentation are used for continuous improvement of recognition accuracy in emotion estimation AI. Application fields include UX optimization for personal health management services, patient explanation support in medical institutions, risk assessment reports for insurance companies, and public health monitoring for local governments.

The proposal unit can determine the priority of proposals based on the submission timing of the meal kit during proposal. The proposal unit determines the priority of proposals based on the submission timing of the meal kit during proposal. For example, the proposal unit prioritizes recently submitted meal kits. The proposal unit postpones meal kits with older submission timing. The proposal unit adjusts the proposal schedule based on submission timing. As a result, the proposal unit can prioritize and provide the latest information to the user. Specifically, the proposal unit receives meal kit data with metadata including submission date and timestamp information (e.g., ISO8601 date/time, UNIX timestamp, etc.) as input data. The proposal unit automatically calculates priority scores for each submission timing, assigns high priority to the latest data, and low priority to older data. Based on the priority scores, the proposal unit uses proposal scheduling algorithms (e.g., priority queue, round robin, FIFO, etc.) to automatically determine the proposal order. Examples of input include “Submission date/time=2024-06-20 10:00”, “Submission date/time=2024-05-15 09:30”, etc. The proposal unit prioritizes meal kits with newer submission timing, and examples of output include “Priority proposal: 2024-06-20 meal kit”, “Postponed: 2024-05-15 meal kit”, etc. In subsequent processing, prioritized proposal content is immediately linked to the delivery scheduler and user notification module and used for prompt feedback and intervention proposals to the user. As a technical effect, unlike conventional uniform proposal order or manual scheduling, the proposal unit can realize rapid proposal of the latest data and timely information provision to the user by AI-based submission timing analysis and dynamic priority control, greatly improving the real-time nature of health management and user satisfaction. Furthermore, by accumulating submission timing data, continuous learning of the AI model and improvement of scheduling accuracy are possible. Application fields include real-time proposals for personal health food subscription services, dietary guidance for patients in medical institutions, health intervention programs for insurance companies, and health promotion measures for local governments.

The proposal unit can adjust the order of proposals based on the relevance of the meal kit during proposal. The proposal unit adjusts the order of proposals based on the relevance of the meal kit during proposal. For example, the proposal unit prioritizes proposals for highly relevant meal kits. The proposal unit postpones proposals for meal kits with low relevance. The proposal unit adjusts the proposal schedule based on the relevance of the meal kit. As a result, the proposal unit can prioritize and provide highly relevant information to the user. Specifically, the proposal unit receives relevance scores for each meal kit (e.g., contribution to disease risk reduction, degree of match with user's health goals, past satisfaction scores, etc.) and the user's health status and lifestyle data as input data. The proposal unit quantifies the relevance between meal kits using multilayer perceptrons or clustering algorithms for relevance estimation (e.g., k-means, topic modeling). Examples of input include “Meal kit A (relevance=0.91)”, “Meal kit B (relevance=0.12)”, etc. The proposal unit adds meal kits with high relevance scores to the proposal target list with priority, while meal kits with low relevance are postponed. The proposal unit executes a proposal order determination algorithm (e.g., descending sort by relevance score, priority queue) to automate proposal scheduling. Examples of output include “Priority proposal: low-carb Japanese-style menu”, “Postponed: gluten-free meal kit”, etc. In subsequent processing, prioritized proposal content is linked to the delivery scheduler and user notification module and used as evidence data for individually optimized meal proposals and health intervention plans. As a technical effect, unlike conventional uniform proposal order or manual relevance judgment, the proposal unit can efficiently and accurately extract and propose truly important information for the user by AI-based relevance estimation and dynamic order optimization, greatly improving the usefulness of health management and user satisfaction. Furthermore, by accumulating relevance scores and proposal history, continuous learning of the AI model and improvement of proposal accuracy are possible. Application fields include enhancement of personalization for personal health food subscription services, dietary guidance for patients in medical institutions, health intervention programs for insurance companies, and health promotion measures for local governments.

The voice input unit can estimate the user's emotions and adjust the timing of voice input based on the estimated emotions. The voice input unit estimates the user's emotions and adjusts the timing of voice input based on the estimated emotions. For example, the voice input unit prompts detailed voice input when the user is relaxed. The voice input unit prompts concise voice input when the user feels stressed. The voice input unit prompts rapid voice input when the user is in a hurry. Emotion estimation is realized by using emotion estimation functions, such as emotion engines or generative AI. Generative AI includes text generation AI (e.g., LLM) and multimodal generative AI, but is not limited to these examples. As a result, the voice input unit can perform voice input at the optimal timing according to the user's emotions. Specifically, the voice input unit receives voice data obtained from the user terminal (e.g., 16 kHz, 16 bit PCM waveform), text input (e.g., “I am calm now”, “I am in a hurry”, etc.), and facial images (e.g., 224×224 pixel RGB images) as input data for emotion estimation AI. The voice input unit can use a multimodal neural network combining a Transformer-based model for speech recognition, a CNN for facial expression recognition, and a large language model for text emotion classification as the emotion estimation AI. Examples of input include “Speech: slow utterance”, “Text: calm today”, “Image: smile”, etc. The voice input unit extracts acoustic features (e.g., MFCC, pitch, energy), text embedding vectors (e.g., BERT-based 768-dimensional vector), and image feature maps (e.g., ResNet output 512-dimensional vector), and outputs emotion categories (e.g., stress, relaxation, hurry, etc.) and emotion scores (e.g., stress level 0.80, relaxation level 0.15, etc.) in the integration layer. Examples of output include “Emotion=relaxation”, “Emotion=stress”, “Emotion=hurry”, etc. Based on these emotion estimation results, the voice input unit executes a voice input timing control algorithm (e.g., detailed input prompt during relaxation, simple input prompt during stress, one-tap input UI during hurry). In subsequent processing, the determined timing and input prompts are linked to the user terminal's UI and notification module, contributing to optimization of user experience and improvement of input completion rate. As a technical effect, unlike conventional uniform input timing prompts or user-dependent operations, the voice input unit can reduce the user's psychological and time burden and greatly improve the completion rate and data quality of voice input by AI-based multimodal emotion estimation and dynamic timing control. Furthermore, cross-entropy loss and data augmentation (e.g., adding noise to speech, rotating/changing brightness of facial images) are used for continuous improvement of recognition accuracy in emotion estimation AI. Application fields include UX optimization for personal health management services, patient input support in medical institutions, data collection efficiency improvement for insurance companies, and health data collection support for local governments.

The voice input unit can analyze the user's past voice input history during voice input and select the optimal input method. The voice input unit analyzes the user's past voice input history during voice input and selects the optimal input method. For example, the voice input unit preferentially proposes voice input methods previously used by the user. The voice input unit selects the most efficient method based on the user's past voice input history. The voice input unit automatically selects methods that the user has previously used without problems. As a result, the voice input unit can provide the optimal voice input method for the user. Specifically, the voice input unit receives a voice input history database for each user (e.g., input date/time, device type used, input method (e.g., continuous speech, keyword input, voice command, etc.), number of errors, required time, etc. recorded as structured data) as input data. The voice input unit can use a history analysis module combining LSTM or Transformer Encoder for time series analysis and clustering algorithms (e.g., k-means, DBSCAN). Examples of input include history records such as “2024/06/01 20:15 continuous speech success”, “2024/06/10 08:30 keyword input failure”, “2024/06/15 21:00 voice command success”, etc. The voice input unit extracts features (e.g., success rate, average required time, error frequency, user operation tendency) from each history and calculates efficiency scores and reliability scores for each input method (e.g., continuous speech=0.92, keyword=0.65, voice command=0.98, etc.). Examples of output include “Recommended method=voice command”, “Recommended method=continuous speech”, “Recommended method =keyword”, etc. Based on these AI outputs, the voice input unit automatically switches the UI of the user terminal (e.g., default display of voice command UI, emphasis on continuous speech button) or optimizes the order of input method options. In subsequent processing, the selected input method is linked to the input execution module and user notification module, contributing to optimization of user experience and reduction of errors. As a technical effect, unlike conventional uniform input method prompts or user-dependent selection, the voice input unit can automatically provide the optimal voice input experience tailored to each user's operational proficiency and environment by AI-based history analysis and dynamic UI optimization, greatly improving input success rate and user satisfaction. Furthermore, by accumulating history data, continuous learning of the AI model and improvement of personalization accuracy for input methods are possible. Application fields include personal health management services, data linkage support in medical institutions, automated risk assessment for insurance companies, and health data collection infrastructure for local governments.

The voice input unit can estimate the user's emotions and determine the priority of voice input based on the estimated emotions. The voice input unit estimates the user's emotions and determines the priority of voice input based on the estimated emotions. For example, the voice input unit prioritizes important voice input when the user feels stressed. The voice input unit sequentially performs all voice input when the user is relaxed. The voice input unit performs only the most important voice input when the user is in a hurry. Emotion estimation is realized by using emotion estimation functions, such as emotion engines or generative AI. Generative AI includes text generation AI (e.g., LLM) and multimodal generative AI, but is not limited to these examples. As a result, the voice input unit can perform voice input with optimal priority according to the user's emotions. Specifically, the voice input unit receives voice data obtained from the user terminal (e.g., 16 kHz, 16 bit PCM format), text input (e.g., “I am in a hurry today”, “I am calm now”, etc.), and facial images (e.g., 224×224 pixel RGB images) as input data for emotion estimation AI. The voice input unit can use a multimodal neural network integrating a Transformer for speech, CNN for facial expression, and a large language model for text. Examples of input include “Speech: fast”, “Text: in a hurry”, “Image: frown”, etc. The voice input unit extracts features from each modality and outputs emotion categories (e.g., stress, relaxation, hurry, etc.) and emotion scores (e.g., stress level 0.80, hurry level 0.65, etc.). Examples of output include “Emotion=stress”, “Emotion=relaxation”, “Emotion=hurry”, etc. The voice input unit combines these emotion estimation results with importance information for voice input items (e.g., priority labels for health status reports, medication records, lifestyle changes, etc.) and executes a priority determination algorithm (e.g., only top-priority items during stress, all items during relaxation, only the most important item during hurry). Examples of output include “Priority list=[health status report, medication record]”, “Input target=top 2 important items”, etc. In subsequent processing, the determined priority is linked to the input execution module and user notification module and reflected in the input UI and progress display. As a technical effect, unlike conventional uniform input order or user-dependent selection, the voice input unit can reduce the user's psychological and time burden and realize rapid acquisition of important data and improvement of input completion rate by AI-based emotion estimation and dynamic priority control. Furthermore, cross-entropy loss and utilization of history data are used for continuous improvement of accuracy in emotion estimation AI and priority determination algorithms. Application fields include personal health management services, emergency data collection in medical institutions, automated risk assessment for insurance companies, and health data collection support for local governments.

The voice input unit can prioritize highly relevant input by considering the user's geographic location information during voice input. The voice input unit prioritizes highly relevant input by considering the user's geographic location information during voice input. For example, the voice input unit prioritizes voice input related to the region when the user is in a specific area. The voice input unit prioritizes voice input related to the current location when the user is traveling. The voice input unit prioritizes voice input related to the new address when the user is planning to move. As a result, the voice input unit can perform optimal voice input based on the user's geographic location information. Specifically, the voice input unit receives geographic location information obtained from the user terminal's GPS or IP address (e.g., latitude/longitude pair, prefecture/municipality code, country code, etc.) as input data. Furthermore, voice input items are provided with metadata for region-specific health information and environmental factor-related input (e.g., pollen allergy symptom report, UV protection, region-specific dietary habit report, etc.). The voice input unit can use graph neural networks for estimating the relevance between geographic information and input items or rule-based regional mapping algorithms. Examples of input include “Location=Shinjuku, Tokyo”, “Location=Sapporo, Hokkaido”, “Location=California, USA”, etc. The voice input unit calculates region-relatedness scores between the current or planned location and voice input items (e.g., pollen allergy report=high, UV protection=medium, etc.) and outputs a priority input list (e.g., top region-relatedness input list). Examples of output include “Priority input=[pollen allergy symptom report, UV protection]”, “Region-relatedness=high”, etc. Based on these AI outputs, the voice input unit automatically determines the target data and order for input and optimizes the UI and notification content of the user terminal. In subsequent processing, prioritized input data is linked to the analysis unit and determination unit and used for region-specific health risk assessment and intervention proposals. As a technical effect, unlike conventional uniform data input or data acquisition ignoring regions, the voice input unit can realize prioritized acquisition of data tailored to region-specific health risks and environmental factors by AI-based integrated analysis of geographic information and input items, greatly improving the accuracy and regional suitability of health management. Furthermore, by accumulating geographic information and input data, continuous learning of the AI model and advancement of region-specific services are possible. Application fields include regional optimization for personal health management services, regional epidemiology support in medical institutions, regional risk assessment for insurance companies, and public health measures for local governments.

The visualization unit can estimate the user's emotions and adjust the display method of visualization based on the estimated emotions. The visualization unit estimates the user's emotions and adjusts the display method of visualization based on the estimated emotions. For example, the visualization unit provides detailed visualization when the user is relaxed. The visualization unit provides concise visualization when the user feels stressed. The visualization unit provides visually appealing visualization when the user is excited. Emotion estimation is realized by using emotion estimation functions, such as emotion engines or generative AI. Generative AI includes text generation AI (e.g., LLM) and multimodal generative AI, but is not limited to these examples. As a result, the visualization unit can provide visualization using the optimal display method according to the user's emotions. Specifically, the visualization unit receives voice data obtained from the user terminal (e.g., 16 kHz, 16 bit PCM waveform), text input (e.g., “I am calm today”, “I am busy now”, etc.), and facial images (e.g., 224×224 pixel RGB images) as input data for emotion estimation AI. The visualization unit can use a multimodal neural network combining a Transformer-based model for speech recognition, a CNN for facial expression recognition, and a large language model for text emotion classification as the emotion estimation AI. The visualization unit extracts acoustic features (e.g., MFCC, pitch, energy), text embedding vectors (e.g., BERT-based 768-dimensional vector), and image feature maps (e.g., ResNet output 512-dimensional vector), and outputs emotion categories (e.g., stress, relaxation, excitement, etc.) and emotion scores (e.g., stress level 0.82, relaxation level 0.15, etc.) in the integration layer. Examples of input include “Speech: slow utterance”, “Text: calm today”, “Image: smile”, etc. Based on the output of the emotion estimation AI, the visualization unit dynamically switches the display method of visualization. For example, when the relaxation level is high, a dashboard including detailed graphs, infographics, and explanations of causal relationships is generated; when the stress level is high, only the main points are presented in bullet points; and when the excitement level is high, interactive visual displays using colors and animations are provided. When determining the display method of visualization, the visualization unit also considers the user's past browsing history and preferences to realize a personalized UI/UX. The visualization unit automatically generates output formats for visualization results, such as text summaries, detailed graphs, infographics, dashboards, etc. Examples of output include “Type 2 diabetes risk=0.78, main cause: rs1234567 (T/T), high-fat diet (with detailed graph)”, “Hypertension risk=0.45 (main points only)”, etc. In subsequent processing, the output of the visualization unit is linked to the user interface and proposal unit and used for feedback and intervention proposals optimized for the user's emotional state. As a technical effect, unlike conventional uniform graph display or manual adjustment of presentation, the visualization unit can automatically generate visualization results tailored to the user's psychological state and situation in real time by AI-based multimodal emotion estimation and dynamic display optimization, greatly improving user understanding, acceptance, and behavior change rates. Furthermore, cross-entropy loss and data augmentation (e.g., adding noise to speech, rotating/changing brightness of facial images) are used for continuous improvement of recognition accuracy in emotion estimation AI. Application fields include UX optimization for personal health management services, patient explanation support in medical institutions, risk assessment reports for insurance companies, and public health monitoring for local governments.

The visualization unit can refer to the user's past visualization history during visualization and select the optimal display method. The visualization unit refers to the user's past visualization history during visualization and selects the optimal display method. For example, the visualization unit preferentially proposes visualization methods previously used by the user. The visualization unit selects the most efficient method based on the user's past visualization history. The visualization unit automatically selects methods that the user has previously used without problems. As a result, the visualization unit can provide the optimal visualization method for the user. Specifically, the visualization unit receives a visualization history database for each user (e.g., display date/time, device type used, display method (e.g., graph, infographic, dashboard, etc.), user operation history, satisfaction score, etc. recorded as structured data) as input data. The visualization unit can use a history analysis module combining LSTM or Transformer Encoder for time series analysis and clustering algorithms (e.g., k-means, DBSCAN). Examples of input include history records such as “2024/06/01 20:15 graph display success”, “2024/06/10 08:30 infographic display failure”, “2024/06/15 21:00 dashboard display success”, etc. The visualization unit extracts features (e.g., display success rate, average viewing time, user operation tendency, satisfaction score) from each history and calculates efficiency scores and reliability scores for each display method (e.g., graph=0.92, infographic=0.65, dashboard=0.98, etc.). Examples of output include “Recommended method=dashboard”, “Recommended method=graph”, “Recommended method=infographic”, etc. Based on these AI outputs, the visualization unit automatically switches the UI of the user terminal (e.g., default display of dashboard UI, emphasis on graph button) or optimizes the order of display method options. In subsequent processing, the selected display method is linked to the display execution module and user notification module, contributing to optimization of user experience and improvement of understanding. As a technical effect, unlike conventional uniform display method prompts or user-dependent selection, the visualization unit can automatically provide the optimal visualization experience tailored to each user's operational proficiency and environment by AI-based history analysis and dynamic UI optimization, greatly improving display success rate and user satisfaction. Furthermore, by accumulating history data, continuous learning of the AI model and improvement of personalization accuracy for display methods are possible. Application fields include personal health management services, data visualization support in medical institutions, risk assessment reports for insurance companies, and public health monitoring for local governments.

The visualization unit can estimate the user's emotions and determine the priority of visualization based on the estimated emotions. The visualization unit estimates the user's emotions and determines the priority of visualization based on the estimated emotions. For example, the visualization unit prioritizes important visualization when the user feels stressed. The visualization unit sequentially performs all visualization when the user is relaxed. The visualization unit performs only the most important visualization when the user is in a hurry. Emotion estimation is realized by using emotion estimation functions, such as emotion engines or generative AI. Generative AI includes text generation AI (e.g., LLM) and multimodal generative AI, but is not limited to these examples. As a result, the visualization unit can perform visualization with optimal priority according to the user's emotions. Specifically, the visualization unit receives voice data obtained from the user terminal (e.g., 16 kHz, 16 bit PCM format), text input (e.g., “I am in a hurry today”, “I am calm now”, etc.), and facial images (e.g., 224×224 pixel RGB images) as input data for emotion estimation AI. The visualization unit can use a multimodal neural network integrating a Transformer for speech, CNN for facial expression, and a large language model for text. Examples of input include “Speech: fast”, “Text: in a hurry”, “Image: frown”, etc. The visualization unit extracts features from each modality and outputs emotion categories (e.g., stress, relaxation, hurry, etc.) and emotion scores (e.g., stress level 0.80, hurry level 0.65, etc.). Examples of output include “Emotion=stress”, “Emotion=relaxation”, “Emotion=hurry”, etc. The visualization unit combines these emotion estimation results with importance information for visualization items (e.g., disease risk visualization, lifestyle change graph, etc. priority labels) and executes a priority determination algorithm (e.g., only top-priority items during stress, all items during relaxation, only the most important item during hurry). Examples of output include “Priority list=[disease risk graph, lifestyle change]”, “Display target=top 2 important items”, etc. In subsequent processing, the determined priority is linked to the display execution module and user notification module and reflected in the display UI and progress display. As a technical effect, unlike conventional uniform display order or user-dependent selection, the visualization unit can reduce the user's psychological and time burden and realize rapid visualization of important data and improvement of understanding by AI-based emotion estimation and dynamic priority control. Furthermore, cross-entropy loss and utilization of history data are used for continuous improvement of accuracy in emotion estimation AI and priority determination algorithms. Application fields include personal health management services, emergency data visualization in medical institutions, automated risk assessment for insurance companies, and health data monitoring for local governments.

The visualization unit can select the optimal display method during visualization by considering the user's device information. The visualization unit selects the optimal display method during visualization by taking into account the user's device information. For example, when the user is using a smartphone, the visualization unit provides a display method tailored to the screen size. When the user is using a tablet, the visualization unit provides a display method optimized for a larger screen. When the user is using a smartwatch, the visualization unit provides a concise and highly visible display method. Thus, the visualization unit can provide the optimal display method based on the user's device information. Specifically, the visualization unit receives device information (e.g., device type, screen resolution, OS version, input interface type, etc. as structured data) obtained from the user terminal as input data. The visualization unit can use rule-based algorithms or decision tree models for device information analysis, or device-adaptive UI generation AI. Examples of input include “device=smartphone, resolution=1080×2400”, “device=tablet, resolution=2048×1536”, “device=smartwatch, resolution=390×390”, and so on. The visualization unit automatically selects the display method (e.g., vertical graph for smartphones, multi-panel dashboard for tablets, icon+numeric display for smartwatches) according to device type and screen size, and optimizes the layout of UI components, font size, and interaction method. Examples of output include “display method=graph for smartphone”, “display method=dashboard for tablet”, “display method=icon for smartwatch”, etc. As a subsequent process, the selected display method is linked to the UI rendering module and user notification module, contributing to the optimization of user experience and improvement of visibility. As a technical effect, unlike conventional uniform display methods or manual device determination, the visualization unit can automatically provide the optimal visualization experience suited to the user's usage environment through AI-based device information analysis and dynamic UI optimization, thereby greatly improving display success rate and user satisfaction. Furthermore, by accumulating device information and display history, continuous learning of the AI model and improvement of personalization accuracy for display methods are possible. Application fields include multi-device support for personal health management services, data visualization support for medical institutions, risk assessment reports for insurance companies, and public health monitoring for local governments.

The improvement unit can estimate the user's emotions and adjust the method of improvement based on the estimated emotions. The improvement unit estimates the user's emotions and adjusts the method of improvement according to the estimated emotions. For example, when the user is relaxed, the improvement unit provides detailed improvement methods. When the user is feeling stressed, the improvement unit provides concise improvement methods. When the user is excited, the improvement unit provides visually attractive improvement methods. Emotion estimation is realized by using emotion estimation functions, such as emotion engines or generative AI. Generative AI may include text generation AI (e.g., LLM) or multimodal generative AI, but is not limited to these examples. Thus, the improvement unit can perform improvement in the optimal manner according to the user's emotions. Specifically, the improvement unit receives input data such as voice data obtained from the user terminal (e.g., 16 kHz, 16 bit PCM format audio waveform), text input (e.g., “I am calm today”, “I am busy now” as natural language sentences), and facial images (e.g., 224×224 pixel RGB images) for the emotion estimation AI. The improvement unit can use a multimodal neural network combining a Transformer-based model for voice recognition, a CNN for facial expression recognition, and a large language model for text emotion classification as the emotion estimation AI. The improvement unit extracts acoustic features (e.g., MFCC, pitch, energy), text embedding vectors (e.g., 768-dimensional BERT-based vectors), and image feature maps (e.g., 512-dimensional vectors output by ResNet), and outputs emotion categories (e.g., stress, relaxation, excitement) and emotion scores (e.g., stress level 0.82, relaxation level 0.15) in the integration layer. Examples of input include “voice: slow speech”, “text: I am calm today”, “image: smile”, etc. Based on the output of the emotion estimation AI, the improvement unit dynamically switches the expression and content of improvement methods. For example, when the relaxation level is high, it generates an improvement report including detailed procedures, scientific basis, and implementation examples; when the stress level is high, it presents only the key points in a bulleted list; when the excitement level is high, it provides interactive visual improvement proposals using colors and animations. In determining the improvement method, the improvement unit also considers the user's past feedback history and preferences to realize personalized UI/UX. The improvement unit automatically generates output formats for improvement results, such as text summaries, detailed reports, infographics, and dashboards. Examples of output include “sleep habit improvement plan (with detailed explanation)”, “exercise habit improvement (key points only)”, etc. As a subsequent process, the output of the improvement unit is linked to the user notification module and progress management module, and is used for feedback and intervention proposals optimized for the user's emotional state. As a technical effect, unlike conventional uniform improvement content presentation or manual expression adjustment, the improvement unit can automatically generate improvement content suited to the user's psychological state and situation in real time through AI-based multimodal emotion estimation and dynamic expression optimization, thereby greatly improving user understanding, acceptance, and behavior change rate. Furthermore, cross-entropy loss and data augmentation (e.g., adding noise to voice, rotating or changing brightness of facial images) are used for learning the emotion estimation AI, enabling continuous improvement of recognition accuracy. Application fields include UX optimization for personal health management services, patient explanation support for medical institutions, health intervention programs for insurance companies, and health promotion measures for local governments.

The improvement unit can refer to the user's past feedback history during improvement to select the optimal improvement method. The improvement unit refers to the user's past feedback history during improvement to select the optimal improvement method. For example, the improvement unit proposes the optimal improvement method based on feedback previously provided by the user. The improvement unit selects the most efficient improvement method from the user's past feedback history. The improvement unit automatically selects improvement methods that the user has previously accepted without problems. Thus, the improvement unit can provide the optimal improvement method for the user. Specifically, the improvement unit receives a feedback history database for each user (e.g., structured data recording improvement proposal ID, implementation date and time, user evaluation score, implementation completion flag, recurrence rate, satisfaction comments, etc.) as input data. The improvement unit can use a history analysis AI module that combines a time-series neural network for history analysis (e.g., LSTM, Transformer Encoder), clustering algorithms (e.g., k-means, DBSCAN), and a large language model for natural language processing of feedback content. The improvement unit extracts features from history data (e.g., success rate for each past improvement method, average satisfaction score, recurrence rate, degree of user behavior change, positive/negative judgment of feedback text, etc.). Based on these features, the improvement unit calculates efficiency scores and reliability scores for each improvement method (e.g., sleep habit improvement=0.92, exercise habit improvement=0.85, dietary habit improvement=0.78, etc.). Examples of input records include “2024/06/01 sleep habit improvement success satisfaction=5”, “2024/06/10 exercise habit improvement failure satisfaction=2”, “2024/06/15 dietary habit improvement success satisfaction=4”, etc. The improvement unit generates AI outputs such as “recommended improvement method=sleep habit improvement”, “recommended improvement method=dietary habit improvement”, and preferentially proposes improvement methods for which the user has shown high satisfaction in the past. Furthermore, the improvement unit also considers user attribute information (e.g., age, gender, lifestyle vector), current health status, and recent feedback content to automatically select personalized improvement methods. As a subsequent process, the selected improvement method is linked to the user notification module and progress management module, contributing to the optimization of user experience and improvement of behavior change rate. As a technical effect, unlike conventional uniform improvement method presentation or manual history reference, the improvement unit can automatically generate optimal improvement proposals suited to each user's behavioral tendencies and past acceptability in real time through AI-based history analysis and dynamic improvement method optimization, thereby greatly improving implementation rate, satisfaction, and health outcomes. Furthermore, by accumulating history data and improvement results, continuous learning of the AI model and improvement of personalization accuracy for improvement methods are possible. Application fields include support for behavior change in personal health management services, utilization of patient guidance history in medical institutions, optimization of health intervention programs for insurance companies, and individualization of public health measures for local governments.

The improvement unit can estimate the user's emotions and determine the priority of improvement based on the estimated emotions. The improvement unit estimates the user's emotions and determines the priority of improvement according to the estimated emotions. For example, when the user is feeling stressed, the improvement unit prioritizes important improvements. When the user is relaxed, the improvement unit performs all improvements sequentially. When the user is in a hurry, the improvement unit performs only the most important improvement. Emotion estimation is realized by using emotion estimation functions, such as emotion engines or generative AI. Generative AI may include text generation AI (e.g., LLM) or multimodal generative AI, but is not limited to these examples. Thus, the improvement unit can perform improvement with the optimal priority according to the user's emotions. Specifically, the improvement unit receives input data such as voice data obtained from the user terminal (e.g., 16 kHz, 16 bit PCM format), text input (e.g., “I am in a hurry today”, “I am calm now”, etc.), and facial images (e.g., 224×224 pixel RGB images) for the emotion estimation AI. The improvement unit can use a multimodal neural network combining a Transformer-based model for voice recognition, a CNN for facial expression recognition, and a large language model for text emotion classification. The improvement unit extracts acoustic features (e.g., MFCC, pitch, energy), text embedding vectors (e.g., 768-dimensional BERT-based vectors), and image feature maps (e.g., 512-dimensional vectors output by ResNet), and outputs emotion categories (e.g., stress, relaxation, hurry) and emotion scores (e.g., stress level 0.80, hurry level 0.65) in the integration layer. Examples of input include “voice: fast speech”, “text: in a hurry”, “image: frown”, etc. The improvement unit combines the output of the emotion estimation AI with importance information for each improvement item (e.g., sleep habit improvement=0.92, exercise habit improvement=0.85, dietary habit improvement=0.78, etc.), and executes a priority determination algorithm (e.g., during stress, only top importance items; during relaxation, all items; during hurry, only the most important item). The improvement unit generates AI outputs such as “priority list=[sleep habit improvement, exercise habit improvement]”, “implementation targets=top 2 important items”, and automatically determines the priority of improvement proposals suited to the user's psychological state and situation. As a subsequent process, the determined priority is linked to the user notification module and progress management module, and reflected in the improvement implementation UI and progress display. As a technical effect, unlike conventional uniform improvement order or manual priority adjustment, the improvement unit can reduce the user's psychological and time burden and realize rapid implementation of important improvement items and improvement of behavior change rate through AI-based multimodal emotion estimation and dynamic priority control. Furthermore, cross-entropy loss and utilization of history data are used for learning the emotion estimation AI and priority determination algorithm, enabling continuous improvement of accuracy. Application fields include support for behavior change in personal health management services, prioritization of patient guidance in medical institutions, optimization of health intervention programs for insurance companies, and emergency response in public health measures for local governments.

The improvement unit can select the optimal improvement method during improvement by considering the user's geographic location information. The improvement unit selects the optimal improvement method during improvement by taking into account the user's geographic location information. For example, when the user is in a specific region, the improvement unit prioritizes improvement methods related to that region. When the user is traveling, the improvement unit prioritizes improvement methods related to the current location. When the user is planning to move, the improvement unit prioritizes improvement methods related to the new address. Thus, the improvement unit can provide the optimal improvement method based on the user's geographic location information. Specifically, the improvement unit receives geographic location information obtained from the user terminal's GPS or IP address (e.g., latitude and longitude pairs, prefecture/municipality codes, country codes, etc.) as input data. Furthermore, improvement items are provided with metadata for region-specific health issues and environmental factor-related improvements (e.g., pollen allergy countermeasures, UV countermeasures, region-specific dietary habit improvements, etc.). The improvement unit can use graph neural networks for estimating the relevance between geographic information and improvement items, or rule-based regional mapping algorithms. Examples of input include “location=Shinjuku, Tokyo”, “location=Sapporo, Hokkaido”, “location=California, USA”, etc. The improvement unit calculates regional relevance scores between the current or planned location and improvement items (e.g., pollen allergy countermeasures=high, UV countermeasures=medium), and outputs a prioritized improvement list (e.g., top region-related improvement list). Examples of output include “priority improvements=[pollen allergy countermeasures, UV countermeasures]”, “regional relevance=high”, etc. Based on these AI outputs, the improvement unit automatically determines the improvement target data and order, and optimizes the UI and notification content on the user terminal. As a subsequent process, the prioritized improvement data is linked to the user notification module and progress management module, and utilized for region-specific health risk assessment and intervention proposals. As a technical effect, unlike conventional uniform improvement method presentation or interventions that ignore regional factors, the improvement unit can realize improvement proposals suited to region-specific health risks and environmental factors through AI-based integrated analysis of geographic information and improvement items, greatly improving the accuracy and regional suitability of health management. Furthermore, by accumulating geographic information and improvement data, continuous learning of the AI model and advancement of region-specialized services are possible. Application fields include regional optimization of personal health management services, regional epidemiology support for medical institutions, regional risk assessment for insurance companies, and public health measures for local governments.

The system according to the embodiment is not limited to the examples described above, and various modifications are possible, for example, as follows. Specifically, the system can implement various technical variations in each module for analysis, determination, proposal, visualization, and improvement of genetic test data, such as the type of AI model and algorithm configuration, data flow, input/output specifications, user interface design, database structure, communication protocol, security method, cloud linkage method, distributed processing infrastructure, multi-device support, extended API linkage, external service linkage, learning data augmentation methods, personalization methods, feedback loop design, anomaly detection and alert notification functions, time-series data analysis, real-time inference, batch processing switching, linkage of multiple AI models (e.g., a pipeline configuration of emotion estimation AI+risk determination AI+proposal generation AI), multidimensional data integration of user attributes, geographic information, history information, device information, learning methods such as data augmentation, transfer learning, continual learning, federated learning, UI/UX optimization algorithms, addition of explainable AI (XAI) modules, enhancement of privacy protection, data anonymization and tokenization, API linkage with external medical institutions, insurance companies, and local government systems, internationalization support (multilingual UI, multi-region data support), automatic recovery functions in case of failure, authority management for each user group, log audit and traceability enhancement, automatic version management of AI models, A/B testing, online learning, and so on. As a result, the system can flexibly and expansively respond to the latest AI technologies, cloud infrastructure, distributed processing technologies, security requirements, legal regulations, and changes in user needs without depending on specific configurations or algorithms. As a technical effect, unlike conventional fixed health management systems or services dependent on a single AI model, the system can flexibly apply multilayered technical improvements in module configuration, algorithms, data flow, learning methods, UI/UX, external linkage, etc., thereby greatly improving the scalability, maintainability, operational efficiency, personalization accuracy, security, reliability, and user satisfaction of the entire system. Application fields include multipurpose deployment of personal health management services, patient support infrastructure for medical institutions, risk assessment and intervention platforms for insurance companies, public health data infrastructure for local governments, and international health data linkage infrastructure.

The receiving unit can estimate the user's emotions and adjust the timing of uploading genetic test results based on the estimated emotions. For example, when the user is feeling stressed, the receiving unit prompts uploading during a time when the user can relax. When the user is busy, the receiving unit adjusts so that uploading can be completed in a short time. When the user is relaxed, the receiving unit provides detailed explanations during uploading. Thus, the receiving unit can upload genetic test results at the optimal timing according to the user's emotions.

The analysis unit can estimate the user's emotions and adjust the method of presenting analysis based on the estimated emotions. For example, when the user is relaxed, the analysis unit provides detailed analysis results. When the user is feeling stressed, the analysis unit provides concise analysis results. When the user is excited, the analysis unit provides visually attractive analysis results. Thus, the analysis unit can provide analysis results in the optimal manner according to the user's emotions.

The proposal unit can estimate the user's emotions and adjust the method of presenting proposals based on the estimated emotions. For example, when the user is relaxed, the proposal unit provides detailed proposals. When the user is feeling stressed, the proposal unit provides concise proposals. When the user is excited, the proposal unit provides visually attractive proposals. Thus, the proposal unit can provide proposals in the optimal manner according to the user's emotions.

The visualization unit can estimate the user's emotions and adjust the display method of visualization based on the estimated emotions. For example, when the user is relaxed, the visualization unit provides detailed visualization. When the user is feeling stressed, the visualization unit provides concise visualization. When the user is excited, the visualization unit provides visually attractive visualization. Thus, the visualization unit can provide visualization in the optimal display method according to the user's emotions.

The improvement unit can estimate the user's emotions and adjust the method of improvement based on the estimated emotions. For example, when the user is relaxed, the improvement unit provides detailed improvement methods. When the user is feeling stressed, the improvement unit provides concise improvement methods. When the user is excited, the improvement unit provides visually attractive improvement methods. Thus, the improvement unit can perform improvement in the optimal manner according to the user's emotions.

The receiving unit can analyze the user's past upload history at the time of uploading genetic test results and select the optimal upload method. For example, the receiving unit preferentially proposes upload methods (manual, voice input, etc.) that the user has used in the past. The receiving unit selects the most efficient method from the user's past upload history. The receiving unit automatically selects methods that the user has previously uploaded without problems. Thus, the receiving unit can provide the optimal upload method for the user.

The analysis unit can adjust the level of detail of analysis during analysis based on the importance of genetic information. For example, the analysis unit provides detailed analysis results for important genetic information. The analysis unit provides concise analysis results for genetic information of low importance. The analysis unit preferentially analyzes genetic information of high importance based on the user's interest. Thus, the analysis unit can analyze important information in detail and provide it to the user.

The proposal unit can adjust the level of detail of proposals during proposal based on the importance of the meal kit. For example, the proposal unit provides detailed proposals for important meal kits. The proposal unit provides concise proposals for meal kits of low importance. The proposal unit preferentially proposes meal kits of high importance based on the user's interest. Thus, the proposal unit can propose important information in detail and provide it to the user.

The visualization unit can select the optimal display method during visualization by considering the user's device information. For example, when the user is using a smartphone, the visualization unit provides a display method tailored to the screen size. When the user is using a tablet, the visualization unit provides a display method optimized for a larger screen. When the user is using a smartwatch, the visualization unit provides a concise and highly visible display method. Thus, the visualization unit can provide the optimal display method based on the user's device information.

The improvement unit can refer to the user's past feedback history during improvement to select the optimal improvement method. For example, the improvement unit proposes the optimal improvement method based on feedback previously provided by the user. The improvement unit selects the most efficient improvement method from the user's past feedback history. The improvement unit automatically selects improvement methods that the user has previously accepted without problems. Thus, the improvement unit can provide the optimal improvement method for the user.

The following is a brief description of the processing flow of Example of the Embodiment.
Step 1: The receiving unit uploads the genetic test results received by the user. For example, the user collects samples such as saliva or blood using a genetic test kit and sends them to a testing institution. The testing institution analyzes the status of approximately 21,000 genetic sets of the user and provides the results. The receiving unit uploads the genetic test results received by the user to the generative AI.
Step 2: The analysis unit analyzes the uploaded genetic test results and questionnaire information entered by the user. For example, the analysis unit analyzes whether specific genes increase the risk of disease. The analysis unit analyzes questionnaire information about the user's current health status and lifestyle habits to grasp the user's lifestyle habits.
Step 3: The determination unit determines disease risk based on information analyzed by the analysis unit. For example, if specific genes increase the risk of disease, the determination unit determines points for improving diet and lifestyle habits to reduce the risk.
Step 4: The proposal unit proposes a meal kit for reducing the disease risk determined by the determination unit. For example, the proposal unit proposes a nutritionally balanced meal based on the user's genetic information and lifestyle habits. The proposal unit can also propose a meal kit customizable according to the user's preferences.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of the plurality of elements including the aforementioned receiving unit, analysis unit, determination unit, proposal unit, voice input unit, visualization unit, and improvement unit is implemented by at least one of, for example, a smart device 14 and a data processing apparatus 12. For example, the receiving unit is implemented by a control unit 46A of the smart device 14 and enables a user to upload genetic test results. The analysis unit is implemented, for example, by a specific processing unit 290 of the data processing apparatus 12 and analyzes the uploaded genetic test results and questionnaire information. The determination unit is implemented, for example, by the specific processing unit 290 of the data processing apparatus 12 and determines disease risk based on the analyzed information. The proposal unit is implemented, for example, by the control unit 46A of the smart device 14 and proposes a meal kit for reducing disease risk. The voice input unit supports voice input of the questionnaire using, for example, a microphone 38B of the smart device 14. The visualization unit visually displays analysis results using, for example, a display 40A of the smart device 14. The improvement unit is implemented, for example, by the specific processing unit 290 of the data processing apparatus 12, collects user feedback, and improves the proposed content. The correspondence between each unit and the device or control unit is not limited to the examples described above and various modifications are possible.

### Second Embodiment

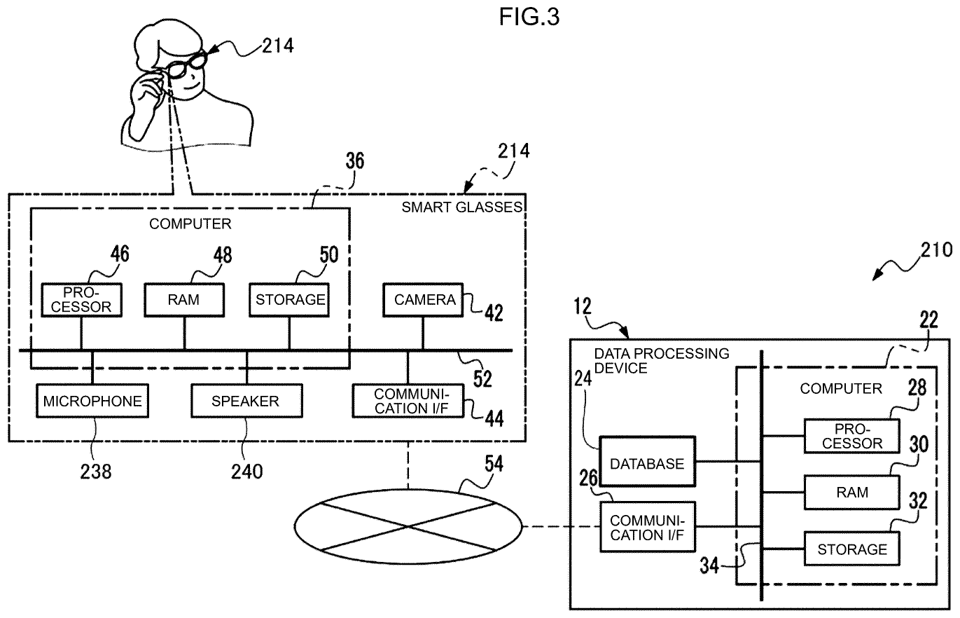

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

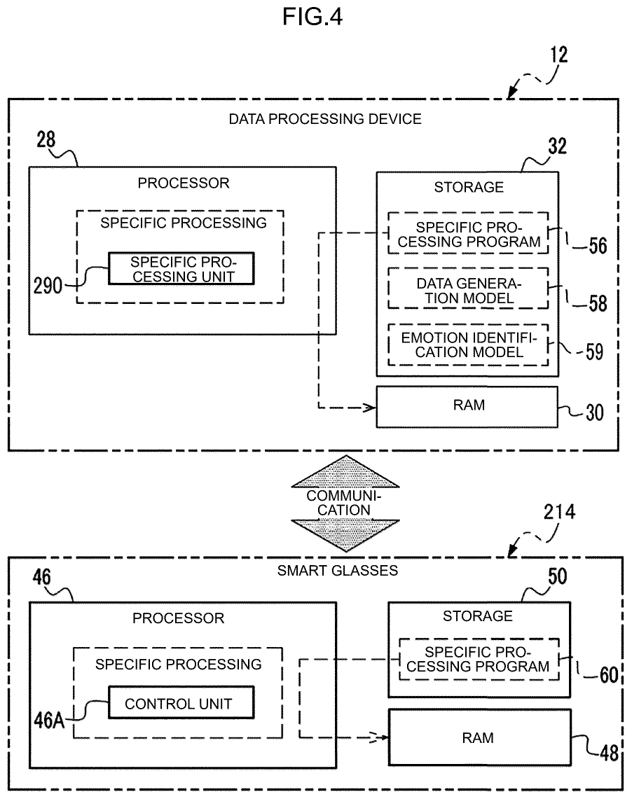

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of the plurality of elements including the aforementioned receiving unit, analysis unit, determination unit, proposal unit, voice input unit, visualization unit, and improvement unit is implemented by at least one of, for example, smart glasses 214 and a data processing apparatus 12. For example, the receiving unit is implemented by a control unit 46A of the smart glasses 214 and enables a user to upload genetic test results. The analysis unit is implemented, for example, by a specific processing unit 290 of the data processing apparatus 12 and analyzes the uploaded genetic test results and questionnaire information. The determination unit is implemented, for example, by the specific processing unit 290 of the data processing apparatus 12 and determines disease risk based on the analyzed information. The proposal unit is implemented, for example, by the control unit 46A of the smart glasses 214 and proposes a meal kit for reducing disease risk. The voice input unit supports voice input of the questionnaire using, for example, a microphone 238 of the smart glasses 214. The visualization unit visually displays analysis results using, for example, a display of the smart glasses 214. The improvement unit is implemented, for example, by the specific processing unit 290 of the data processing apparatus 12, collects user feedback, and improves the proposed content. The correspondence between each unit and the device or control unit is not limited to the examples described above and various modifications are possible.

### Third Embodiment

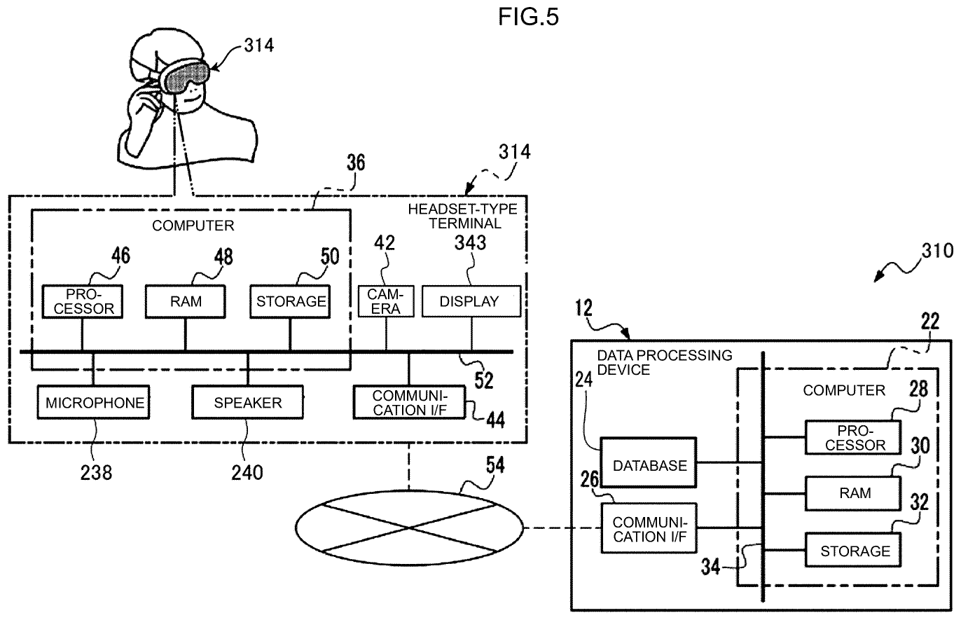

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

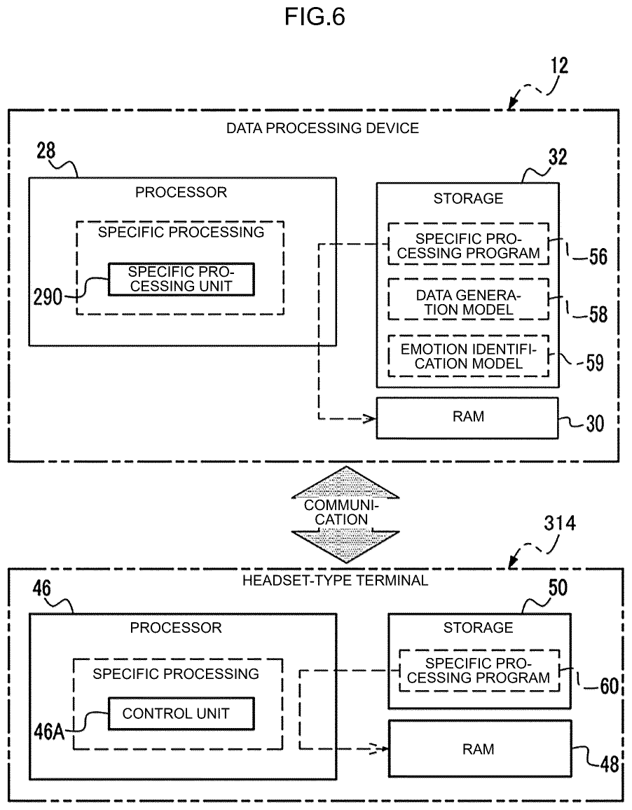

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of the plurality of elements including the aforementioned receiving unit, analysis unit, determination unit, proposal unit, voice input unit, visualization unit, and improvement unit is implemented by at least one of, for example, a headset-type terminal 314 and a data processing apparatus 12. For example, the receiving unit is implemented by a control unit 46A of the headset-type terminal 314 and enables a user to upload genetic test results. The analysis unit is implemented, for example, by a specific processing unit 290 of the data processing apparatus 12 and analyzes the uploaded genetic test results and questionnaire information. The determination unit is implemented, for example, by the specific processing unit 290 of the data processing apparatus 12 and determines disease risk based on the analyzed information. The proposal unit is implemented, for example, by the control unit 46A of the headset-type terminal 314 and proposes a meal kit for reducing disease risk. The voice input unit supports voice input of the questionnaire using, for example, a microphone 238 of the headset-type terminal 314. The visualization unit visually displays analysis results using, for example, a display 343 of the headset-type terminal 314. The improvement unit is implemented, for example, by the specific processing unit 290 of the data processing apparatus 12, collects user feedback, and improves the proposed content. The correspondence between each unit and the device or control unit is not limited to the examples described above and various modifications are possible.

### Fourth Embodiment

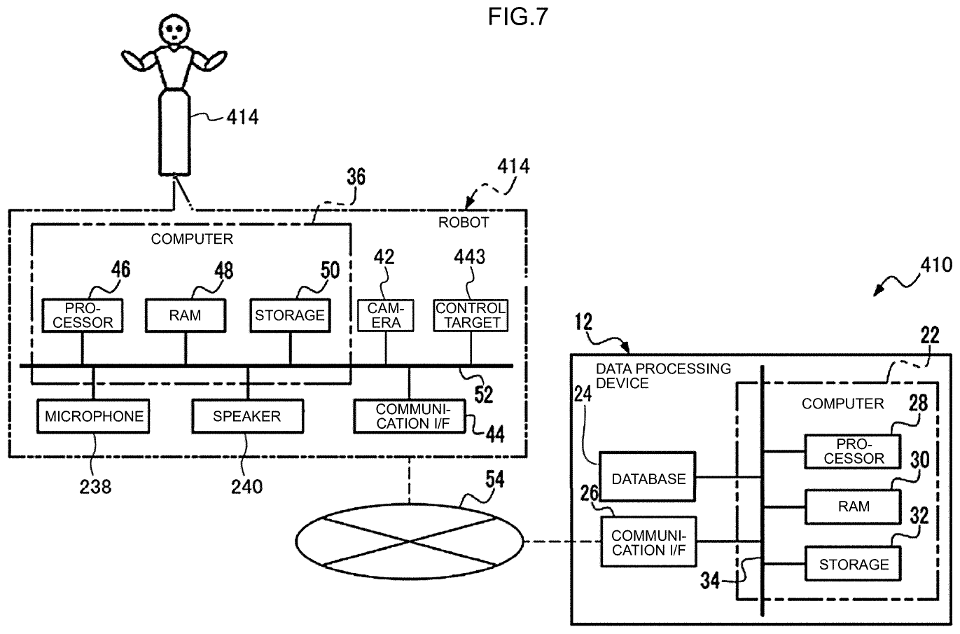

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

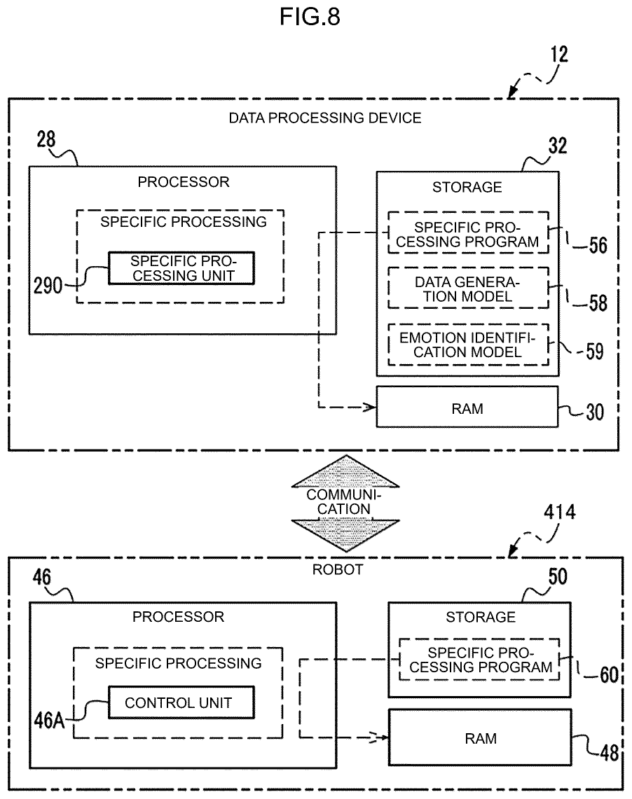

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of the plurality of elements including the aforementioned receiving unit, analysis unit, determination unit, proposal unit, voice input unit, visualization unit, and improvement unit is implemented by at least one of, for example, a robot 414 and a data processing apparatus 12. For example, the receiving unit is implemented by a control unit 46A of the robot 414 and enables a user to upload genetic test results. The analysis unit is implemented, for example, by a specific processing unit 290 of the data processing apparatus 12 and analyzes the uploaded genetic test results and questionnaire information. The determination unit is implemented, for example, by the specific processing unit 290 of the data processing apparatus 12 and determines disease risk based on the analyzed information. The proposal unit is implemented, for example, by the control unit 46A of the robot 414 and proposes a meal kit for reducing disease risk. The voice input unit supports voice input of the questionnaire using, for example, a microphone 238 of the robot 414. The visualization unit visually displays analysis results using, for example, a display of the robot 414. The improvement unit is implemented, for example, by the specific processing unit 290 of the data processing apparatus 12, collects user feedback, and improves the proposed content. The correspondence between each unit and the device or control unit is not limited to the examples described above and various modifications are possible.

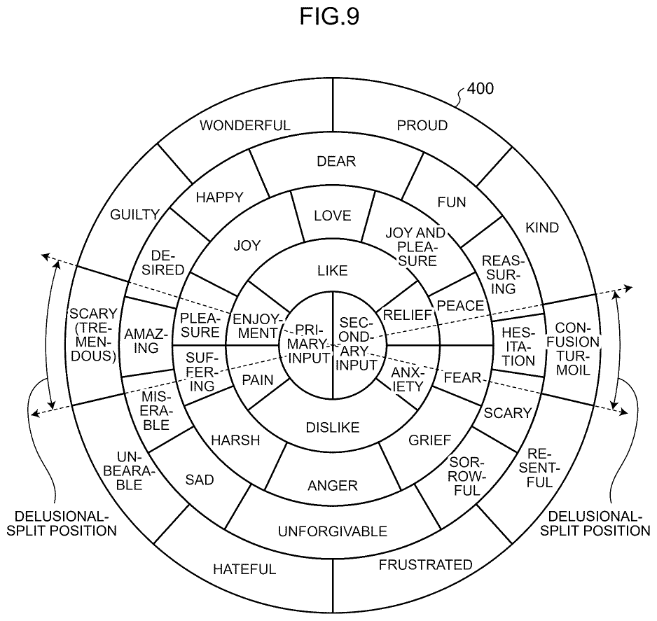

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

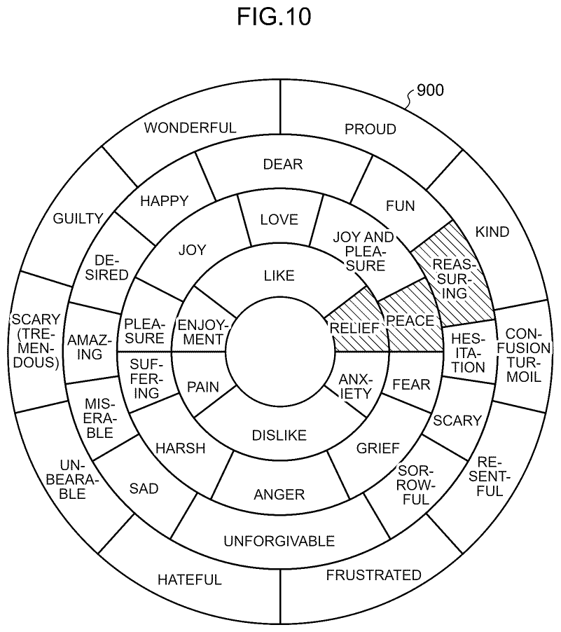

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.
(Supplementary Note 1)A system comprising: a receiving unit configured to upload genetic test results; an analysis unit configured to analyze the genetic test results uploaded by the receiving unit and questionnaire information; a determination unit configured to determine disease risk based on information analyzed by the analysis unit; and a proposal unit configured to propose a meal kit for reducing the disease risk determined by the determination unit.
(Supplementary Note 2)The system according to Supplementary Note 1, further comprising a unit configured to enable voice input for entering the questionnaire.
(Supplementary Note 3)The system according to Supplementary Note 1, further comprising a unit configured to visualize analysis results.
(Supplementary Note 4)The system according to Supplementary Note 1, further comprising a unit configured to improve the proposed content based on user feedback.
(Supplementary Note 5)The system according to Supplementary Note 1, wherein the receiving unit comprises a unit configured to automatically upload genetic test results.
(Supplementary Note 6)The system according to Supplementary Note 1, wherein the analysis unit comprises a unit configured to analyze genetic information and questionnaire information and to grasp the user's lifestyle habits.
(Supplementary Note 7)The system according to Supplementary Note 1, wherein the proposal unit comprises a unit configured to propose a meal kit customizable according to the user's preferences.
(Supplementary Note 8)The system according to Supplementary Note 1, wherein the receiving unit comprises a unit configured to estimate the user's emotions and adjust the timing of uploading genetic test results based on the estimated emotions.
(Supplementary Note 9)The system according to Supplementary Note 1, wherein the receiving unit analyzes the user's past upload history at the time of uploading genetic test results and selects an upload method.
(Supplementary Note 10)The system according to Supplementary Note 1, wherein the receiving unit comprises a unit configured to perform filtering at the time of uploading genetic test results based on the user's current health status and lifestyle habits.
(Supplementary Note 11)The system according to Supplementary Note 1, wherein the receiving unit is configured to estimate the user's emotions and determine the priority of genetic test results to be uploaded based on the estimated emotions.
(Supplementary Note 12)The system according to Supplementary Note 1, wherein the receiving unit is configured to consider the user's geographic location information at the time of uploading genetic test results and preferentially upload highly relevant results.
(Supplementary Note 13)The system according to Supplementary Note 1, wherein the receiving unit is configured to analyze the user's social media activity at the time of uploading genetic test results and upload relevant results.
(Supplementary Note 14)The system according to Supplementary Note 1, wherein the analysis unit is configured to estimate the user's emotions and adjust the method of presenting analysis based on the estimated emotions.
(Supplementary Note 15)The system according to Supplementary Note 1, wherein the analysis unit is configured to adjust the level of detail of analysis based on the importance of genetic information during analysis.
(Supplementary Note 16)The system according to Supplementary Note 1, wherein the analysis unit is configured to apply different analysis algorithms according to the category of genetic information during analysis.
(Supplementary Note 17)The system according to Supplementary Note 1, wherein the analysis unit is configured to estimate the user's emotions and adjust the length of analysis based on the estimated emotions.
(Supplementary Note 18)The system according to Supplementary Note 1, wherein the analysis unit is configured to determine the priority of analysis based on the submission timing of genetic information during analysis.
(Supplementary Note 19)The system according to Supplementary Note 1, wherein the analysis unit is configured to adjust the order of analysis based on the relevance of genetic information during analysis.
(Supplementary Note 20)The system according to Supplementary Note 1, wherein the determination unit is configured to estimate the user's emotions and adjust the criteria for determining disease risk based on the estimated emotions.
(Supplementary Note 21)The system according to Supplementary Note 1, wherein the determination unit is configured to improve the accuracy of determination by considering the interrelationships of genetic information during determination.
(Supplementary Note 22)The system according to Supplementary Note 1, wherein the determination unit is configured to perform determination by considering attribute information of the submitter of genetic information during determination.
(Supplementary Note 23)The system according to Supplementary Note 1, wherein the determination unit is configured to estimate the user's emotions and adjust the display order of determination results based on the estimated emotions.
(Supplementary Note 24)The system according to Supplementary Note 1, wherein the determination unit is configured to perform determination by considering the geographic distribution of genetic information during determination.
(Supplementary Note 25)The system according to Supplementary Note 1, wherein the determination unit is configured to improve the accuracy of determination by referring to related literature of genetic information during determination.
(Supplementary Note 26)The system according to Supplementary Note 1, wherein the proposal unit is configured to estimate the user's emotions and adjust the method of presenting proposals based on the estimated emotions.
(Supplementary Note 27)The system according to Supplementary Note 1, wherein the proposal unit is configured to adjust the level of detail of proposals based on the importance of the meal kit during proposal.
(Supplementary Note 28)The system according to Supplementary Note 1, wherein the proposal unit is configured to apply different proposal algorithms according to the category of the meal kit during proposal.
(Supplementary Note 29)The system according to Supplementary Note 1, wherein the proposal unit is configured to estimate the user's emotions and adjust the length of proposals based on the estimated emotions.
(Supplementary Note 30)The system according to Supplementary Note 1, wherein the proposal unit is configured to determine the priority of proposals based on the submission timing of the meal kit during proposal.
(Supplementary Note 31)The system according to Supplementary Note 1, wherein the proposal unit is configured to adjust the order of proposals based on the relevance of the meal kit during proposal.
(Supplementary Note 32)The system according to Supplementary Note 2, wherein the voice input unit is configured to estimate the user's emotions and adjust the timing of voice input based on the estimated emotions.
(Supplementary Note 33)The system according to Supplementary Note 2, wherein the voice input unit is configured to analyze the user's past voice input history during voice input and select an optimal input method.
(Supplementary Note 34)The system according to Supplementary Note 2, wherein the voice input unit is configured to estimate the user's emotions and determine the priority of voice input based on the estimated emotions.
(Supplementary Note 35)The system according to Supplementary Note 2, wherein the voice input unit is configured to consider the user's geographic location information during voice input and preferentially perform highly relevant input.
(Supplementary Note 36)The system according to Supplementary Note 3, wherein the visualization unit is configured to estimate the user's emotions and adjust the display method of visualization based on the estimated emotions.
(Supplementary Note 37)The system according to Supplementary Note 3, wherein the visualization unit is configured to refer to the user's past visualization history during visualization and select an optimal display method.
(Supplementary Note 38)The system according to Supplementary Note 3, wherein the visualization unit is configured to estimate the user's emotions and determine the priority of visualization based on the estimated emotions.
(Supplementary Note 39)The system according to Supplementary Note 3, wherein the visualization unit is configured to consider the user's device information during visualization and select an optimal display method.
(Supplementary Note 40)The system according to Supplementary Note 4, wherein the improvement unit is configured to estimate the user's emotions and adjust the method of improvement based on the estimated emotions.
(Supplementary Note 41)The system according to Supplementary Note 4, wherein the improvement unit is configured to refer to the user's past feedback history during improvement and select an optimal improvement method.
(Supplementary Note 42)The system according to Supplementary Note 4, wherein the improvement unit is configured to estimate the user's emotions and determine the priority of improvement based on the estimated emotions.
(Supplementary Note 43)The system according to Supplementary Note 4, wherein the improvement unit is configured to consider the user's geographic location information during improvement and select an optimal improvement method.

What is claimed is:

1. A system comprising:
circuitry configured to:
receive, from a client terminal via a communication interface and a packet-switched network, structured data comprising sequence vectors and user attribute data;
store the structured data in a database;
analyze the structured data by inputting the structured data into a data generation model obtained by deep learning on a neural network to generate inference data comprising a risk score and a factor contribution vector;
generate, using the data generation model, recommendation data based on the inference data; and
transmit the recommendation data to the client terminal via the communication interface and the packet-switched network, the recommendation data causing the client terminal to present the recommendation data to a user.

2. The system according to claim 1, wherein the structured data comprises genetic sequence data including single nucleotide polymorphism vectors for a plurality of genetic loci, and wherein the user attribute data comprises at least one of lifestyle habit data, dietary pattern data, or health status data.

3. The system according to claim 1, wherein the circuitry is further configured to preprocess the structured data by performing at least one of missing value imputation, normalization, or anomaly detection before storing the structured data in the database.

4. The system according to claim 1, wherein the data generation model comprises at least one of a Transformer-based large language model, a multilayer perceptron, or a multimodal neural network configured to integrate the sequence vectors and the user attribute data as input tensors.

5. The system according to claim 1, wherein the inference data further comprises a classification label for each of a plurality of categories, and wherein the circuitry is further configured to perform threshold determination based on the risk score to assign a priority level to each classification label.

6. The system according to claim 1, wherein the circuitry is further configured to estimate an emotion of the user by applying an emotion identification model to at least one of voice data, a face image, or text input received from the client terminal, and to adjust a timing of receiving the structured data based on the estimated emotion.

7. The system according to claim 6, wherein the circuitry is further configured to adjust a method of presenting the recommendation data based on the estimated emotion, such that when the estimated emotion indicates relaxation, detailed recommendation data is generated, and when the estimated emotion indicates stress, concise recommendation data is generated.

8. The system according to claim 1, wherein the circuitry is further configured to analyze a past data reception history of the user stored in the database and select an optimal data reception method based on efficiency scores calculated from the past data reception history.

9. The system according to claim 1, wherein the circuitry is further configured to determine a priority of the structured data to be received based on an estimated emotion of the user, such that when the estimated emotion indicates stress, structured data having a high importance attribute is prioritized.

10. The system according to claim 1, wherein the circuitry is further configured to receive geographic location information of the user from the client terminal and preferentially receive structured data associated with a geographic region corresponding to the geographic location information.

11. The system according to claim 1, wherein the circuitry is further configured to receive social media activity data of the user from the client terminal, analyze the social media activity data using a natural language processing model to extract interest topics, and filter the structured data to be received based on the extracted interest topics.

12. The system according to claim 1, wherein the circuitry is further configured to adjust a level of detail of the analysis based on an importance score assigned to the structured data, such that for structured data having a high importance score, a multi-stage analysis flow using a plurality of AI models is applied.

13. The system according to claim 1, wherein the circuitry is further configured to apply different analysis algorithms according to a category of the structured data.

14. The system according to claim 1, wherein the circuitry is further configured to improve accuracy of the inference data by considering interrelationships among elements of the structured data using at least one of a graph neural network or a Bayesian network.

15. The system according to claim 1, wherein the circuitry is further configured to adjust a length of the recommendation data based on an estimated emotion of the user, such that when the estimated emotion indicates urgency, concise recommendation data is generated, and when the estimated emotion indicates relaxation, detailed recommendation data is generated.

16. The system according to claim 1, wherein the circuitry is further configured to determine a priority of generating the recommendation data based on a submission timing associated with the structured data, such that structured data having a more recent submission timing is processed with a higher priority.

17. The system according to claim 1, wherein the circuitry is further configured to receive feedback data from the client terminal, analyze the feedback data using a natural language processing model to extract improvement indicators, and adjust parameters of the data generation model based on the improvement indicators.

18. A system comprising:
a communication interface configured to communicate, via a packet-switched network conforming to at least one of a 5G, Wi-Fi, or Bluetooth communication standard, with a client terminal comprising a touch panel, a microphone, a speaker, a camera having a CMOS image sensor, and a display;
a processor;
a random-access memory;
a storage storing a data generation model obtained by deep learning on a neural network, and an emotion identification model;
a database; and
circuitry configured to:
receive, from the client terminal via the communication interface, structured data comprising sequence vectors and user attribute data, and store the structured data in the database;
estimate an emotion of the user by applying the emotion identification model to at least one of voice data captured by the microphone or image data captured by the camera;
analyze the structured data by inputting the structured data into the data generation model to generate inference data comprising a risk score and a factor contribution vector;
generate, using the data generation model, recommendation data based on the inference data, the recommendation data being adapted based on the estimated emotion;
adjust at least one of a level of detail, an expression style, or a length of the recommendation data based on the estimated emotion; and
transmit the recommendation data to the client terminal via the communication interface, the recommendation data causing the client terminal to present the recommendation data to the user via at least one of the display or the speaker.

19. The system according to claim 18, wherein the data generation model comprises at least one of a text generation AI, an image generation AI, or a multimodal generation AI, and wherein the data generation model is a fine-tuned model configured to output inference results from prompts without instructions.

20. A method performed by circuitry of a system comprising a communication interface, a database, and a storage storing a data generation model obtained by deep learning on a neural network, the method comprising:
receiving, from a client terminal via the communication interface and a packet-switched network, structured data comprising sequence vectors and user attribute data;
storing the structured data in the database;
analyzing the structured data by inputting the structured data into the data generation model to generate inference data comprising a risk score and a factor contribution vector;
generating, using the data generation model, recommendation data based on the inference data; and
transmitting the recommendation data to the client terminal via the communication interface and the packet-switched network, the recommendation data causing the client terminal to present the recommendation data to a user.
